SEQUENCE LISTING (48 sequences)

SEQ ID NO: 1 - DNA, 396 bp, Aspergillus kawachii
  CDS (1)..(396): A. kawachii CBD
actagtacatccaaagccaccacctcctcttcttcttcttctgctgctgctactacttcttcatcatgcaccgcaacaagcaccaccctccccatcaccttcgaagaactcgtcaccactacctacggggaagaagtctacctcagcggatctatctcccagctcggagagtgggatacgagtgacgcggtgaagttgtccgcggatgattatacctcgagtaaccccgagtggtctgttactgtgtcgttgccggtggggacgaccttcgagtataagtttattaaggtcgatgagggtggaagtgtgacttgggaaagtgatccgaatagggagtatactgtgcctgaatgtgggaatgggagtggggagacggtggttgatacgtggaggtag

SEQ ID NO: 2 - protein, 131 aa, Aspergillus kawachii
TSTSKATTSSSSSSAAATTSSSCTATSTTLPITFEELVTTTYGEEVYLSGSISQLGEWDTSDAVKLSADDYTSSNPEWSVTVSLPVGTTFEYKFIKVDEGGSVTWESDPNREYTVPECGNGSGETVVDTWR

SEQ ID NO: 3 - DNA, 1533 bp, Aspergillus niger
  CDS (1)..(1533): Aspergillus niger acid alpha-amylase
atgagattatcgacttcgagtctcttcctttccgtgtctctgctggggaagctggccctcgggctgtcggctgcagaatggcgcactcagtcgatttacttcctattgacggatcggttcggtaggacggacaattcgacgacagctacatgcgatacgggtgaccaaatctattgtggtggcagttggcaaggaatcatcaaccatctggattatatccagggcatgggattcacggccatctggatctcgcctatcactgaacagctgccccaggatactgctgatggtgaagcttaccatggatattggcagcagaagatatacgacgtgaactccaacttcggcactgcagatgacctcaagtccctctcagatgcgcttcatgcccgcggaatgtacctcatggtggacgtcgtccctaaccacatgggctacgccggcaacggcaacgatgtagactacagcgtcttcgaccccttcgattcctcctcctacttccacccatactgcctgatcacagattgggacaacttgaccatggtccaagattgttgggagggtgacaccatcgtatctctgccagacctaaacaccaccgaaactgccgtgagaacaatctggtatgactgggtagccgacctggtatccaattattcagtcgacggactccgcatcgacagtgtcctcgaagtcgaaccagacttcttcccgggctaccaggaagcagcaggtgtctactgcgtcggcgaagtcgacaacggcaaccctgccctcgactgcccataccagaaggtcctggacggcgtcctcaactatccgatctactggcaactcctctacgccttcgaatcctccagcggcagcatcagcaatctctacaacatgatcaaatccgtcgcaagcgactgctccgatccgacactactcggcaacttcatcgaaaaccacgacaatccccgtttcgcctcctacacctccgactactcgcaagccaaaaacgtcctcagctacatcttcctctccgacggcatccccatcgtctacgccggcgaagaacagcactactccggcggcaaggtgccctacaaccgcgaagcgacctggctttcaggctacgacacctccgcagagctgtacacctggatagccaccacgaacgcgatccgcaaactagccatctcagctgactcggcctacattacctacgcgaatgatgcattctacactgacagcaacaccatcgcaatgcgcaaaggcacctcagggagccaagtcatcaccgtcctctccaacaaaggctcctcaggaagcagctacaccctgaccctcagcggaagcggctacacatccggcacgaagctgatcgaagcgtacacatgcacatccgtgaccgtggactcgagcggcgatattcccgtgccgatggcgtcgggattaccgagagttcttctgcccgcgtccgtcgtcgatagctcttcgctctgtggcgggagcggaagaacaaccacgaccacaactgctgctactagt

SEQ ID NO: 4 - protein, 511 aa, Aspergillus niger
MRLSTSSLFLSVSLLGKLALGLSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRTTTTTTAATS

SEQ ID NO: 5 - protein, 102 aa, Bacillus flavothermus
  DOMAIN (1)..(102): CBD
ISTTSQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVP

SEQ ID NO: 6 - protein, 99 aa, Bacillus sp.
  DOMAIN (1)..(99): CBD
TSNVTFTVNNATTVYGQNVYVVGNIPELGNWNIANAIQMTPSSYPTWKTTVSLPQGKAIEFKFIKKDSAGNVIWENIANRTYTVPFSSTGSYTANWNVP

SEQ ID NO: 7 - protein, 102 aa, Bacillus
  DOMAIN (1)..(102): CBD
TSTTSQITFTVNNATTVWGQNVYVVGNISQLGNWDPVNAVQMTPSSYPTWVVTVPLPQSQNIQFKFIKKDGSGNVIWENISNRTYTVPTAASGAYTANWNVP

SEQ ID NO: 8 - protein, 112 aa, Hormoconis resinae
  DOMAIN (1)..(112): CBD
CQVSITFNINATTYYGENLYVIGNSSDLGAWNIADAYPLSASAYTQDRPLWSAAIPLNAGEVISYQYVRQEDCDQPYIYETVNRTLTVPACGGAAVTTDDAWMGPVGSSGNC

SEQ ID NO: 9 - protein, 95 aa, Lentinula edodes
  DOMAIN (1)..(95): CBD
VSVTFNVDASTLEGQNVYLTGAVDALEDWSTDNAILLSSANYPTWSVTVDLPGSTDVQYKYIKKDGSGTVTWESDPNMEITTPANGTYATNDTWR

SEQ ID NO: 10 - protein, 107 aa, Neurospora crassa
  DOMAIN (1)..(107): CBD
CAADHEVLVTFNEKVTTSYGQTVKVVGSIAALGNWAPASGVTLSAKQYSSSNPLWSTTIALPQGTSFKYKYVVVNSDGSVKWENDPDRSYAVGTDCASTATLDDTWR

SEQ ID NO: 11 - protein, 115 aa, Talaromyces byssochlamydioides
  DOMAIN (1)..(115): CBD
TTTGAAPCTTPTTVAVTFDEIVTTTYGETVYLSGSIPALGNWDTSSAIALSAVDYTSSNPLWYVTVNLPAGTSFEYKFFVQQTDGTIVWEDDPNRSYTVPANCGQTTAIIDDSWQ

SEQ ID NO: 12 - protein, 115 aa, Geosmithia cylindrospora
  DOMAIN (1)..(115): CBD
TSTGSAPCTTPTTVAVTFDEIVTTSYGETVYLAGSIAALGNWDTNSAIALSAADYTSNNNLWYVTVNLAAGTSFQYKFFVKETDSTIVWEDDPNRSYTVPANCGQTTAIIDDTWQ

SEQ ID NO: 13 - protein, 139 aa, Scorias spongiosa
  DOMAIN (1)..(139): CBD
AKVPSTCSASSATGTCTTATSTFGGSTPTTSCATTPTLTTVLFNERATTNFGQNVHLTGSISQLGSWDTDSAVALSAVNYTSSDPLWFVRVQLPAGTSFQYKYFKKDSSNAVAWESDPNRSYTVPLNCAGTATENDTWR

SEQ ID NO: 14 - protein, 126 aa, Eupenicillium ludwigii
  DOMAIN (1)..(126): CBD
STTTTSTTKTTTTSTTTSCTTPTAVAVTFDLIATTYYGENIKIAGSISQLGDWDTSNAVALSAADYTSSDHLWFVDIDLPAGTVFEYKYIRIESDGSIEWESDPNRSYTVPAACATTAVTENDTWR

SEQ ID NO: 15 - protein, 116 aa, Aspergillus japonicus
  DOMAIN (1)..(116): CBD
KTSTTTSSCSTPTSVAVTFDVIATTTYGENVYISGSISQLGSWDTSSAIALSASQYTSSNNLWYATVHLPAGTTFQYKYIRKETDGSVTWESDPNRSYTVPSSCGVSSATESDTWR

SEQ ID NO: 16 - protein, 133 aa, Penicillium cf. miczynskii
  DOMAIN (1)..(133): CBD
TTTGGTTTSQGSTTTTSKTSTTTSSCTAPTSVAVTFDLIATTVYDENVQLAGSISALGSWDTSSAIRLSASQYTSSNHLWYVAVSLPAGQVFQYKYIRVASSGTITWESDPNLSYTVPVACAATAVTISDTWR

SEQ ID NO: 17 - protein, 116 aa, Mz1 Penicillium sp.
  DOMAIN (1)..(116): CBD
TKTSTSTSCTTPTAVAVTFDLIATTTYGENIKIAGSIAALGAWDTDDAVALSAADYTDSDHLWFVTQSIPAGTVFEYKYIRVESDGTIEWESDPNRSYTVPAACATTAVTESDTWR

SEQ ID NO: 18 - protein, 114 aa, Thysanophora sp
  DOMAIN (1)..(114): CBD
FTSTTKTSCTTPTSVAVTFDLIATTTYGESIRLVGSISELGDWDTGSAIALHATDYTDSDHLWFVTVGLPAGASFEYKYIRVESSGTIEWESDPNRSYTVPAACATTAVTESDT

SEQ ID NO: 19 - protein, 111 aa, Humicola grisea var. thermoidea
  DOMAIN (1)..(111): CBD
ADASEVYVTFNERVSTAWGETIKVVGNVPALGNWDTSKAVTLSASGYKSNDPLWSITVPIKATGSAVQYKYIKVGTNGKITWESDPNRSITLQTASSAGKCAAQTVNDSWR

SEQ ID NO: 20 - protein, 108 aa, Aspergillus niger [sequence omitted]

SEQ ID NO: 21 - protein, 97 aa, Aspergillus rolfsii
  DOMAIN (1)..(97): CBD
VEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 22 - protein, 38 aa, Aspergillus niger
  DOMAIN (1)..(38): Linker
TGGTTTTATPTGSGSVTSTSKTTATASKTSTSTSSTSA

SEQ ID NO: 23 - protein, 31 aa, Aspergillus kawachii
  DOMAIN (1)..(31): Linker
TTTTTTAAATSTSKATTSSSSSSAAATTSSS

SEQ ID NO: 24 - protein, 11 aa, Athelia rolfsii
  DOMAIN (1)..(11): Linker
GATSPGGSSGS

SEQ ID NO: 25 - protein, 8 aa, Artificial Sequence
  Synthetic
PEPTPEPT

SEQ ID NO: 26 - protein, 498 aa, Aspergillus oryzae
  SIGNAL (1)..(20)
  mat_peptide (20)..(498)
MVAWWSLFLYGLQVAAPALAATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 27 - DNA, 1860 bp, Artificial Sequence
  Synthetic
ctgtcggctgcagaatggcgcactcagtcgatttacttcctattgacggatcggttcggtaggacggacaattcgacgacagctacatgcgatacgggtgaccaaatctattgtggtggcagttggcaaggaatcatcaaccatctggattatatccagggcatgggattcacggccatctggatctcgcctatcactgaacagctgccccaggatactgctgatggtgaagcttaccatggatattggcagcagaagatatacgacgtgaactccaacttcggcactgcagatgacctcaagtccctctcagatgcgcttcatgcccgcggaatgtacctcatggtggacgtcgtccctaaccacatgggctacgccggcaacggcaacgatgtagactacagcgtcttcgaccccttcgattcctcctcctacttccacccatactgcctgatcacagattgggacaacttgaccatggtccaagattgttgggagggtgacaccatcgtatctctgccagacctaaacaccaccgaaactgccgtgagaacaatctggtatgactgggtagccgacctggtatccaattattcagtcgacggactccgcatcgacagtgtcctcgaagtcgaaccagacttcttcccgggctaccaggaagcagcaggtgtctactgcgtcggcgaagtcgacaacggcaaccctgccctcgactgcccataccagaaggtcctggacggcgtcctcaactatccgatctactggcaactcctctacgccttcgaatcctccagcggcagcatcagcaatctctacaacatgatcaaatccgtcgcaagcgactgctccgatccgacactactcggcaacttcatcgaaaaccacgacaatccccgtttcgcctcctacacctccgactactcgcaagccaaaaacgtcctcagctacatcttcctctccgacggcatccccatcgtctacgccggcgaagaacagcactactccggcggcaaggtgccctacaaccgcgaagcgacctggctttcaggctacgacacctccgcagagctgtacacctggatagccaccacgaacgcgatccgcaaactagccatctcagctgactcggcctacattacctacgcgaatgatgcattctacactgacagcaacaccatcgcaatgcgcaaaggcacctcagggagccaagtcatcaccgtcctctccaacaaaggctcctcaggaagcagctacaccctgaccctcagcggaagcggctacacatccggcacgaagctgatcgaagcgtacacatgcacatccgtgaccgtggactcgagcggcgatattcccgtgccgatggcgtcgggattaccgagagttcttctgcccgcgtccgtcgtcgatagctcttcgctctgtggcgggagcggaagaacaaccacgaccacaactgctgctgctactagtacatccaaagccaccacctcctcttcttcttcttctgctgctgctactacttcttcatcatgtaccactcccaccgccgtggctgtgactttcgatctgacagctaccaccacctacggcgagaacatctacctggtcggatcgatctctcagctgggtgactgggaaaccagcgacggcatagctctgagtgctgacaagtacacttccagcgacccgctctggtatgtcactgtgactctgccggctggtgagtcgtttgagtacaagtttatccgcattgagagcgatgactccgtggagtgggagagtgatcccaaccgagaatacaccgttcctcaggcgtgcggaacgtcgaccgcgacggtgactgacacctggcggtag

SEQ ID NO: 28 - protein, 619 aa, Artificial Sequence
  Synthetic Construct
LSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRTTTTTTAAATSTSKATTSSSSSSAAATTSSSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 29 - DNA, 1827 bp, Artificial Sequence
  Synthetic
ctgtcggctgcagaatggcgcactcagtcgatttacttcctattgacggatcggttcggtaggacggacaattcgacgacagctacatgcgatacgggtgaccaaatctattgtggtggcagttggcaaggaatcatcaaccatctggattatatccagggcatgggattcacggccatctggatctcgcctatcactgaacagctgccccaggatactgctgatggtgaagcttaccatggatattggcagcagaagatatacgacgtgaactccaacttcggcactgcagatgacctcaagtccctctcagatgcgcttcatgcccgcggaatgtacctcatggtggacgtcgtccctaaccacatgggctacgccggcaacggcaacgatgtagactacagcgtcttcgaccccttcgattcctcctcctacttccacccatactgcctgatcacagattgggacaacttgaccatggtccaagattgttgggagggtgacaccatcgtatctctgccagacctaaacaccaccgaaactgccgtgagaacaatctggtatgactgggtagccgacctggtatccaattattcagtcgacggactccgcatcgacagtgtcctcgaagtcgaaccagacttcttcccgggctaccaggaagcagcaggtgtctactgcgtcggcgaagtcgacaacggcaaccctgccctcgactgcccataccagaaggtcctggacggcgtcctcaactatccgatctactggcaactcctctacgccttcgaatcctccagcggcagcatcagcaatctctacaacatgatcaaatccgtcgcaagcgactgctccgatccgacactactcggcaacttcatcgaaaaccacgacaatccccgtttcgcctcctacacctccgactactcgcaagccaaaaacgtcctcagctacatcttcctctccgacggcatccccatcgtctacgccggcgaagaacagcactactccggcggcaaggtgccctacaaccgcgaagcgacctggctttcaggctacgacacctccgcagagctgtacacctggatagccaccacgaacgcgatccgcaaactagccatctcagctgactcggcctacattacctacgcgaatgatgcattctacactgacagcaacaccatcgcaatgcgcaaaggcacctcagggagccaagtcatcaccgtcctctccaacaaaggctcctcaggaagcagctacaccctgaccctcagcggaagcggctacacatccggcacgaagctgatcgaagcgtacacatgcacatccgtgaccgtggactcgagcggcgatattcccgtgccgatggcgtcgggattaccgagagttcttctgcccgcgtccgtcgtcgatagctcttcgctctgtggcgggagcggaagaacaaccacgaccacaactgctgctgctactagtacatccaaagccaccacctcctcttcttcttcttctgctgctgctactacttcttcatcagtcgaggtcactttcgacgtttacgctaccacagtatatggccagaacatctatatcaccggtgatgtgagtgagctcggcaactggacacccgccaatggtgttgcactctcttctgctaactaccccacctggagtgccacgatcgctctccccgctgacacgacaatccagtacaagtatgtcaacattgacggcagcaccgtcatctgggaggatgctatcagcaatcgcgagatcacgacgcccgccagcggcacatacaccgaaaaagacacttgggatgaatcttag

SEQ ID NO: 30 - protein, 608 aa, Artificial Sequence
  Synthetic Construct
LSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRTTTTTTAAATSTSKATTSSSSSSAAATTSSSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 31 - DNA, 1863 bp, Artificial Sequence
  Synthetic
gcaacgcctgcggactggcgatcgcaatccatttatttccttctcacggatcgatttgcaaggacggatgggtcgacgactgcgacttgtaatactgcggatcagaaatactgtggtggaacatggcagggcatcatcgacaagttggactatatccagggaatgggcttcacagccatctggatcacccccgttacagcccagctgccccagaccaccgcatatggagatgcctaccatggctactggcagcaggatatatactctctgaacgaaaactacggcactgcagatgacttgaaggcgctctcttcggcccttcatgagagggggatgtatcttatggtcgatgtggttgctaaccatatgggctatgatggagcgggtagctcagtcgattacagtgtgtttaaaccgttcagttcccaagactacttccacccgttctgtttcattcaaaactatgaagatcagactcaggttgaggattgctggctaggagataacactgtctccttgcctgatctcgataccaccaaggatgtggtcaagaatgaatggtacgactgggtgggatcattggtatcgaactactccattgacggcctccgtatcgacacagtaaaacacgtccagaaggacttctggcccgggtacaacaaagccgcaggcgtgtactgtatcggcgaggtgctcgacggtgatccggcctacacttgtccctaccagaacgtcatggacggcgtactgaactatcccatttactatccactcctcaacgccttcaagtcaacctccggcagcatggacgacctctacaacatgatcaacaccgtcaaatccgactgtccagactcaacactcctgggcacattcgtcgagaaccacgacaacccacggttcgcttcttacaccaacgacatagccctcgccaagaacgtcgcagcattcatcatcctcaacgacggaatccccatcatctacgccggccaagaacagcactacgccggcggaaacgaccccgcgaaccgcgaagcaacctggctctcgggctacccgaccgacagcgagctgtacaagttaattgcctccgcgaacgcaatccggaactatgccattagcaaagatacaggattcgtgacctacaagaactggcccatctacaaagacgacacaacgatcgccatgcgcaagggcacagatgggtcgcagatcgtgactatcttgtccaacaagggtgcttcgggtgattcgtataccctctccttgagtggtgcgggttacacagccggccagcaattgacggaggtcattggctgcacgaccgtgacggttggttcggatggaaatgtgcctgttcctatggcaggtgggctacctagggtattgtatccgactgagaagttggcaggtagcaagatctgtagtagctcgggaagaacaaccacgaccacaactgctgctgctactagtacatccaaagccaccacctcctcttcttcttcttctgctgctgctactacttcttcatcatgcaccgcaacaagcaccaccctccccatcaccttcgaagaactcgtcaccactacctacggggaagaagtctacctcagcggatctatctcccagctcggagagtgggatacgagtgacgcggtgaagttgtccgcggatgattatacctcgagtaaccccgagtggtctgttactgtgtcgttgccggtggggacgaccttcgagtataagtttattaaggtcgatgagggtggaagtgtgacttgggaaagtgatccgaatagggagtatactgtgcctgaatgtgggaatgggagtggggagacggtggttgatacgtggaggtag

SEQ ID NO: 32 - protein, 620 aa, Artificial Sequence
  Synthetic Construct
ATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSSGRTTTTTTAAATSTSKATTSSSSSSAAATTSSSCTATSTTLPITFEELVTTTYGEEVYLSGSISQLGEWDTSDAVKLSADDYTSSNPEWSVTVSLPVGTTFEYKFIKVDEGGSVTWESDPNREYTVPECGNGSGETVVDTWR

SEQ ID NO: 33 - DNA, 1767 bp, Artificial Sequence
  Synthetic
ctgtcggctgcagaatggcgcactcagtcgatttacttcctattgacggatcggttcggtaggacggacaattcgacgacagctacatgcgatacgggtgaccaaatctattgtggtggcagttggcaaggaatcatcaaccatctggattatatccagggcatgggattcacggccatctggatctcgcctatcactgaacagctgccccaggatactgctgatggtgaagcttaccatggatattggcagcagaagatatacgacgtgaactccaacttcggcactgcagatgacctcaagtccctctcagatgcgcttcatgcccgcggaatgtacctcatggtggacgtcgtccctaaccacatgggctacgccggcaacggcaacgatgtagactacagcgtcttcgaccccttcgattcctcctcctacttccacccatactgcctgatcacagattgggacaacttgaccatggtccaagattgttgggagggtgacaccatcgtatctctgccagacctaaacaccaccgaaactgccgtgagaacaatctggtatgactgggtagccgacctggtatccaattattcagtcgacggactccgcatcgacagtgtcctcgaagtcgaaccagacttcttcccgggctaccaggaagcagcaggtgtctactgcgtcggcgaagtcgacaacggcaaccctgccctcgactgcccataccagaaggtcctggacggcgtcctcaactatccgatctactggcaactcctctacgccttcgaatcctccagcggcagcatcagcaatctctacaacatgatcaaatccgtcgcaagcgactgctccgatccgacactactcggcaacttcatcgaaaaccacgacaatccccgtttcgcctcctacacctccgactactcgcaagccaaaaacgtcctcagctacatcttcctctccgacggcatccccatcgtctacgccggcgaagaacagcactactccggcggcaaggtgccctacaaccgcgaagcgacctggctttcaggctacgacacctccgcagagctgtacacctggatagccaccacgaacgcgatccgcaaactagccatctcagctgactcggcctacattacctacgcgaatgatgcattctacactgacagcaacaccatcgcaatgcgcaaaggcacctcagggagccaagtcatcaccgtcctctccaacaaaggctcctcaggaagcagctacaccctgaccctcagcggaagcggctacacatccggcacgaagctgatcgaagcgtacacatgcacatccgtgaccgtggactcgagcggcgatattcccgtgccgatggcgtcgggattaccgagagttcttctgcccgcgtccgtcgtcgatagctcttcgctctgtggcgggagcggaagaggtgctacaagcccgggtggctcctcgggtagtgtcgaggtcactttcgacgtttacgctaccacagtatatggccagaacatctatatcaccggtgatgtgagtgagctcggcaactggacacccgccaatggtgttgcactctcttctgctaactaccccacctggagtgccacgatcgctctccccgctgacacgacaatccagtacaagtatgtcaacattgacggcagcaccgtcatctgggaggatgctatcagcaatcgcgagatcacgacgcccgccagcggcacatacaccgaaaaagacacttgggatgaatcttag

SEQ ID NO: 34 - protein, 588 aa, Artificial Sequence
  Synthetic Construct
LSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 35 - DNA, 1767 bp, Artificial Sequence
  Synthetic
gcaacgcctgcggactggcgatcgcaatccatttatttccttctcacggatcgatttgcaaggacggatgggtcgacgactgcgacttgtaatactgcggatcagaaatactgtggtggaacatggcagggcatcatcgacaagttggactatatccagggaatgggcttcacagccatctggatcacccccgttacagcccagctgccccagaccaccgcatatggagatgcctaccatggctactggcagcaggatatatactctctgaacgaaaactacggcactgcagatgacttgaaggcgctctcttcggcccttcatgagagggggatgtatcttatggtcgatgtggttgctaaccatatgggctatgatggagcgggtagctcagtcgattacagtgtgtttaaaccgttcagttcccaagactacttccacccgttctgtttcattcaaaactatgaagatcagactcaggttgaggattgctggctaggagataacactgtctccttgcctgatctcgataccaccaaggatgtggtcaagaatgaatggtacgactgggtgggatcattggtatcgaactactccattgacggcctccgtatcgacacagtaaaacacgtccagaaggacttctggcccgggtacaacaaagccgcaggcgtgtactgtatcggcgaggtgctcgacggtgatccggcctacacttgtccctaccagaacgtcatggacggcgtactgaactatcccatttactatccactcctcaacgccttcaagtcaacctccggcagcatggacgacctctacaacatgatcaacaccgtcaaatccgactgtccagactcaacactcctgggcacattcgtcgagaaccacgacaacccacggttcgcttcttacaccaacgacatagccctcgccaagaacgtcgcagcattcatcatcctcaacgacggaatccccatcatctacgccggccaagaacagcactacgccggcggaaacgaccccgcgaaccgcgaagcaacctggctctcgggctacccgaccgacagcgagctgtacaagttaattgcctccgcgaacgcaatccggaactatgccattagcaaagatacaggattcgtgacctacaagaactggcccatctacaaagacgacacaacgatcgccatgcgcaagggcacagatgggtcgcagatcgtgactatcttgtccaacaagggtgcttcgggtgattcgtataccctctccttgagtggtgcgggttacacagccggccagcaattgacggaggtcattggctgcacgaccgtgacggttggttcggatggaaatgtgcctgttcctatggcaggtgggctacctagggtattgtatccgactgagaagttggcaggtagcaagatctgtagtagctcgggaagaggtgctacaagcccgggtggctcctcgggtagtgtcgaggtcactttcgacgtttacgctaccacagtatatggccagaacatctatatcaccggtgatgtgagtgagctcggcaactggacacccgccaatggtgttgcactctcttctgctaactaccccacctggagtgccacgatcgctctccccgctgacacgacaatccagtacaagtatgtcaacattgacggcagcaccgtcatctgggaggatgctatcagcaatcgcgagatcacgacgcccgccagcggcacatacaccgaaaaagacacttgggatgaatcttag

SEQ ID NO: 36 - protein, 588 aa, Artificial Sequence
  Synthetic Construct
ATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSSGRGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 37 - protein, 640 aa, Aspergillus kawachii
  mat_peptide (22)..(640)
MRVSTSSIALAVSLFGKLALGLSAAEWRTQSIYFLLTDRFGRTDNSTTATCNTGDQIYCGGSWQGIINHLDYIQGMGFTAIWISPITEQLPQDTSDGEAYHGYWQQKIYYVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVEEVEPDFFPGYQEAAGVYCVGEVDNGNPALDCPYQKYLDGVLNYPIYWQLLYAFESSSGSISNLYNMIKSVASDCSDPTLLGNFIENHDNPRFASYTSDYSQAKNVLSYIFLSDGIPIVYAGEEQHYSGGDVPYNREATWLSGYDTSAELYTWIATTNAIRKLAISADSDYITYKNDPIYTDSNTIAMRKGTSGSQIITVLSNKGSSGSSYTLTLSGSGYTSGTKLIEAYTCTSVTVDSNGDIPVPMASGLPRVLLPASVVDSSSLCGGSGNTTTTTTAATSTSKATTSSSSSSAAATTSSSCTATSTTLPITFEELVTTTYGEEVYLSGSISQLGEWHTSDAVKLSADDYTSSNPEWSVTVSLPVGTTFEYKFIKVDEGGSVTWESDPNREYTVPECGSGSGETVVDTWR

SEQ ID NO: 38 - protein, 505 aa, Aspergillus niger
  mat_peptide (22)..(505)
MRLSTSSLFLSVSLLGKLALGLSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRLYVE

SEQ ID NO: 39 - protein, 476 aa, Aspergillus oryzae
  mat_peptide (1)..(476)
ATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICS

SEQ ID NO: 40 - DNA, 1455 bp, Bacillus sp.
  CDS (1)..(1455): AA560
caccataatggtacgaacggcacaatgatgcagtactttgaatggtatctaccaaatgacggaaaccattggaatagattaaggtctgatgcaagtaacctaaaagataaagggatctcagcggtttggattcctcctgcatggaagggtgcctctcaaaatgatgtggggtatggtgcttatgatctgtatgatttaggagaattcaatcaaaaaggaaccattcgtacaaaatatggaacgcgcaatcagttacaagctgcagttaacgccttgaaaagtaatggaattcaagtgtatggcgatgttgtaatgaatcataaagggggagcagacgctaccgaaatggttagggcagttgaagtaaacccgaataatagaaatcaagaagtgtccggtgaatatacaattgaggcttggacaaagtttgactttccaggacgaggtaatactcattcaaacttcaaatggagatggtatcactttgatggagtagattgggatcagtcacgtaagctgaacaatcgaatttataaatttagaggtgatggaaaagggtgggattgggaagtcgatacagaaaacggtaactatgattacctaatgtatgcagatattgacatggatcacccagaggtagtgaatgagctaagaaattggggtgtttggtatacgaatacattaggccttgatggttttagaatagatgcagtaaaacatataaaatacagctttactcgtgattggattaatcatgttagaagtgcaactggcaaaaatatgtttgcggttgcggaattttggaaaaatgatttaggtgctattgaaaactatttaaacaaaacaaactggaaccattcagtctttgatgttccgctgcactataacctctataatgcttcaaaaagcggagggaattatgatatgaggcaaatatttaatggtacagtcgtgcaaagacatccaatgcatgctgttacatttgttgataatcatgattcgcaacctgaagaagctttagagtcttttgttgaagaatggttcaaaccattagcgtatgctttgacattaacacgtgaacaaggctacccttctgtattttatggagattattatggcattccaacgcatggtgtaccagcgatgaaatcgaaaattgacccgattctagaagcgcgtcaaaagtatgcatatggaagacaaaatgactacttagaccatcataatatcatcggttggacacgtgaagggaatacagcacaccccaactccggtttagctactatcatgtccgatggggcaggaggaaataagtggatgtttgttgggcgtaataaagctggtcaagtttggaccgatatcactggaaatcgtgcaggtactgttacgattaatgctgatggatggggtaatttttctgtaaatggaggatcagtttctatttgggtaaacaaa

SEQ ID NO: 41 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 42 - DNA, 1350 bp, Rhizomucor pusillus
  CDS (1)..(1350)
agccctttgccccaacagcagcgatatggcaaaagagcaacttcggatgactggaaaggcaaggccatttatcagctgcttacagatcgatttggccgcgccgatgactcaacaagcaactgctctaatttatccaactactgtggtggtacctacgaaggcattacgaagcatcttgactacatttccggtatgggctttgatgctatctggatatcgccaattcccaagaactcggatggaggctaccacggctactgggctacagatttctaccaactaaacagcaactttggtgatgaatcccagctcaaagcgctcatccaggctgcccatgaacgtgacatgtatgttatgcttgatgtcgtagccaatcatgcaggtcccaccagcaatggctactcgggttacacattcggcgatgcaagtttatatcatcctaaatgcaccatagattacaatgatcagacgtctattgagcaatgctgggttgctgacgagttgcctgatattgacactgaaaattctgacaacgtggccattctcaacgacatcgtctccggctgggtgggtaactatagctttgacggcatccgcattgatactgtcaagcatattcgcaaggacttttggacaggctacgcagaagctgccggcgtattcgcaactggagaggtcttcaatggtgatccggcctacgttggaccttatcaaaagtacctgccatctctcatcaattacccaatgtattacgctttgaacgacgtctttgtatccaaaagcaaaggattcagccgcatcagcgaaatgctaggatcaaatcgcaatgcgtttgaggataccagcgtacttacaacgtttgtagacaaccatgacaatccgcgcttcttgaacagtcaaagcgacaaggctctcttcaagaacgctctcacatacgtactgctaggtgaaggcatcccaattgtgtattatggttctgagcaaggtttcagcggaggagcggatcctgctaaccgtgaagtgctgtggaccaccaattatgatacatccagcgatctctaccaatttatcaagacagtcaacagtgtccgcatgaaaagcaacaaggccgtctacatggatatttatgttggcgacaatgcttacgccttcaagcacggcgatgctttggttgttctcaataactatggatcaggttccacaaaccaagtcagcttcagcgttagtggcaagttcgatagcggcgcaagcctcatggatattgtcagtaacattaccaccacggtgtcctcggatggaacagtcactttcaaccttaaagatggacttccggctatcttcacctctgct

SEQ ID NO: 43 - protein, 450 aa, Rhizomucor pusillus
SPLPQQQRYGKRATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSA

SEQ ID NO: 44 - protein, 558 aa, Artificial Sequence
  Synthetic
SPLPQQQRYGKRATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSAGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 45 - DNA, 1398 bp, Meripilus giganteus
  CDS (1)..(1398)
cgccctactgtctttgacgccggcgcggacgcacactcgctgcatgcccgggccccctccggcagcaaggatgtcatcatccagatgtttgagtggaactgggacagcgtcgctgccgagtgcactaacttcatcggccccgccgggtacggcttcgtgcaagtgagcccgccccaggagaccatccagggcgcgcagtggtggaccgactaccagccggtgtcgtacacgctcactgggaagcggggcgaccgctcccagtttgcgaacatgattactacgtgccacgccgcgggcgtcggcgtgatcgttgacaccatctggaaccacatggcgggcgtcgactccggcacgggtaccgccggctcgtccttcacgcactacaactaccccggcatctaccaaaaccaggactttcaccactgcggcctcgagccgggcgatgacatcgtcaactacgacaacgcggttgaggtccagacctgcgagcttgtcaacctcgctgacctcgccaccgacacggagtatgtgcgcggtcgccttgcccagtacggaaacgacctgctctcgctcggtgccgatggcctgcgtcttgacgcttccaaacacattcctgtgggcgacatcgcgaacatcctgtctcgcctcagtcgctctgtctacatcacccaggaagtcatctttggggccggcgagcccatcacgccgaaccagtacaccgggaacggcgacgttcaggagttccgctacacctctgcgctaaaggacgccttcttgagctcgggcatatccaacctgcaggacttcgaaaaccgtggatgggtacctggctcgggcgccaacgtgttcgtcgtcaaccatgacaccgagcggaacggcgcgtcgctgaacaacaactcgccttcgaacacctacgtcaccgcgacgatcttctcgctcgcacacccgtacggcacgcccacgatcctctcctcgtatgatggcttcacgaacaccgacgccggtgcgccgaacaacaacgtcggcacatgctcgaccagcggtggtgcgaacgggtggctctgccagcaccgctggaccgcgatcgccggcatggtcggcttccgcaacaacgtcggcagcgctgcactcaacaactggcaggccccgcagtcgcagcagattgcgttcggtcgcggcgcacttggcttcgtcgcgatcaacaacgccgactcggcctggtctacgacgttcaccacttccctccccgatggttcctactgcgatgtcatcagcggcaaggcctccggcagtagctgcaccggttcttcgttcaccgtctccggcgggaagctgaccgccacggtgccggcgcgtagcgccatcgccgtgcacaccggtcagaaaggttctggtggt

SEQ ID NO: 46 - protein, 466 aa, Meripilus giganteus
RPTVFDAGADAHSLHARAPSGSKDVIIQMFEWNWDSVAAECTNFIGPAGYGFVQVSPPQETIQGAQWWTDYQPVSYTLTGKRGDRSQFANMITTCHAAGVGVIVDTIWNHMAGVDSGTGTAGSSFTHYNYPGIYQNQDFHHCGLEPGDDIVNYDNAVEVQTCELVNLADLATDTEYVRGRLAQYGNDLLSLGADGLRLDASKHIPVGDIANILSRLSRSVYITQEVIFGAGEPITPNQYTGNGDVQEFRYTSALKDAFLSSGISNLQDFENRGWVPGSGANVFVVNHDTERNGASLNNNSPSNTYVTATIFSLAHPYGTPTILSSYDGFTNTDAGAPNNNVGTCSTSGGANGWLCQHRWTAIAGMVGFRNNVGSAALNNWQAPQSQQIAFGRGALGFVAINNADSAWSTTFTTSLPDGSYCDVISGKASGSSCTGSSFTVSGGKLTATVPARSAIAVHTGQKGSGG

SEQ ID NO: 47 - protein, 574 aa, Artificial Sequence
  Synthetic
RPTVFDAGADAHSLHARAPSGSKDVIIQMFEWNWDSVAAECTNFIGPAGYGFVQVSPPQETIQGAQWWTDYQPVSYTLTGKRGDRSQFANMITTCHAAGVGVIVDTIWNHMAGVDSGTGTAGSSFTHYNYPGIYQNQDFHHCGLEPGDDIVNYDNAVEVQTCELVNLADLATDTEYVRGRLAQYGNDLLSLGADGLRLDASKHIPVGDIANILSRLSRSVYITQEVIFGAGEPITPNQYTGNGDVQEFRYTSALKDAFLSSGISNLQDFENRGWVPGSGANVFVVNHDTERNGASLNNNSPSNTYVTATIFSLAHPYGTPTILSSYDGFTNTDAGAPNNNVGTCSTSGGANGWLCQHRWTAIAGMVGFRNNVGSAALNNWQAPQSQQIAFGRGALGFVAINNADSAWSTTFTTSLPDGSYCDVISGKASGSSCTGSSFTVSGGKLTATVPARSAIAVHTGQKGSGGGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 48 - protein, 583 aa, Artificial Sequence
  Synthetic
ATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

(19) United States
(12) Patent Application Publication
(10) Pub. No.: US 2009/0286302 A1
(43) Pub. Date: Nov. 19, 2009

(54) Desizing and Scouring Process

(75) Inventors: Jiyin Liu, Raleigh, NC (US); Sonja Salmon, Raleigh, NC (US); Guifang Wu, Beijing (CN)

Correspondence Address: NOVOZYMES NORTH AMERICA, INC., 500 FIFTH AVENUE, SUITE 1600, NEW YORK, NY, 10110, US

(73) Assignee: Novosymes A/S, Bagsvaerd (DK)

(21) Appl. No.: 12/302,308

(22) PCT Filed: Jun. 6, 2007
(86) PCT No.: PCT/US07/70485

§ 371 Date: Nov. 25, 2008

Related U.S. Application Data
(60) Provisional application No. 60/815,788, filed on Jun. 21, 2006.

(51) Int. Cl. C12N 9/28

(52) U.S. Cl. 435/202; 435/263

(57) ABSTRACT

The present invention relates to processes for combined desizing and scouring of a sized fabric containing starch or starch derivatives during manufacture of fabric, which process comprises incubating said sized fabric in an aqueous treating solution having a pH in the range between 1 and 7, which aqueous treating solution comprises an acid amylase and at least one other acid enzyme facilitating said other fabric treatment steps. The present invention further relates to compositions used in said processes and the use of said compositions.

### REFERENCE TO A SEQUENCE LISTING

This application contains a Sequence Listing in computer readable form. The computer readable form is incorporated herein by reference.

### FIELD OF THE INVENTION

The present invention relates to combined desizing and scouring processes using acid-amylase and other enzymes such as cellulase, pectinase, lipase, xylanase, protease, etc during manufacture of new fabrics.

### BACKGROUND OF THE INVENTION

The processing of fabric, such as cellulosic material, into material ready for garment manufacture involves several steps: spinning of the fiber into a yarn; construction of woven or knit fabric from the yarn; and subsequent preparation, dyeing and finishing operations. The preparation process, which may involve desizing (for woven goods), scouring, and bleaching, produces a fabric suitable for dyeing or finishing.

WO 2006/002034 (Novozymes) describes simultaneous desizing and scouring process comprising treating fabric with an alkaline alpha-amylase and an alkaline scouring enzyme. Alkaline alpha-amylases are used as auxiliaries in desizing processes to facilitate the removal of starch-containing size which has served as a protective coating on yarns during weaving.

Complete removal of the size coating after weaving is important to ensure optimum results in the subsequent processes in which the fabric is generally scoured, bleached, dyed and/or printed.

After the desizing step it is often desirable to include a demineralization step in order to remove metal ions, such as Mn²⁺, Fe²⁺/Fe³⁺:Cu²⁺ etc., which—if present on the fabric—may result in an uneven bleaching in a later process step or might even make pin-holes in the bleached fabric. Demineralization is typically accomplished by acid precipitation and typically involves addition of acids such as acetic acid or sulphuric acid.

There is a need for improved processes for simultaneous desizing combined with other fabric treatment steps, such as combined desizing and scouring, combined desizing and biopolishing, combined desizing and abrasion and combined desizing and carbonizing etc.

### BRIEF DISCLOSURE OF THE INVENTION

The present invention is directed towards providing processes of desizing sized fabrics during manufacture of especially new fabrics under acid conditions.

In one aspect, the present invention relates to a process for combined desizing and other fabric treatment steps of a sized fabric containing starch or starch derivatives during manufacture of fabric, which process comprises incubating said sized fabric in an aqueous treating solution having a pH in the range between 1 and 7, preferably between 1 and 5, especially between 1 and 4, which aqueous treating solution comprises an acid amylase and at least one other acid enzyme facilitating said other fabric treatment step(s).

Preferably, said other acid enzyme(s), facilitating said other fabric treatment step(s), is (are) acid cellulase, acid pectinase, acid lipase, acid xylanase and/or acid protease. More preferably, the enzyme(s) facilitating said other fabric treatment step(s), is(are) acid pectinase(s).

Preferably, the acid amylase is of bacterial or fungal origin, such as filamentous fungus origin.

Preferably, the acid amylase is derived from a strain of Aspergillus, preferably Aspergillus niger, Aspergillus awamori, Aspergillus oryzae, or Aspergillus kawachii (SEQ ID NO: 37) or a strain of Rhizomucor, preferably Rhizomucor pusillus, or a strain of Meripilus, preferably a strain of Meripilus giganteus. More preferably the Aspergillus acid amylase is the acid Aspergillus niger alpha-amylase disclosed in SEQ ID NO: 38, or a variant thereof. Even more preferably, the acid amylase is the Rhizomucor pusillus alpha-amylase disclosed in SEQ ID NO: 48, or a variant thereof.

Preferably, the bacterial acid amylase is derived from a strain of the genus Bacillus, preferably derived from a strain of Bacillus sp., more preferably a strain of Bacillus licheniformis Bacillus amyloliquefaciens, Bacillus stearothermophilus, Bacillus subtilis, or Bacillus sp., such as Bacillus sp. NCIB 12289, NCIB 12512, NCIB 12513, DSM 9375, DSMZ 12648, DSMZ 12649, KSM AP1378, KSM K36 or KSM K38.

Hybrid alpha-amylase can also use in the present invention. Preferably, the hybrid alpha-amylase could be the amylase consisting of Rhizomucor pusillus alpha-amylase with Aspergillus niger glucoamylase linker and SBD disclosed as V039 in Table 5 in co-pending International Application no. PCT/US05/46725.

Preferably, the acid alpha-amylase is present in a concentration of 1-3,000 AFAU/kg fabric, preferably 10-1,000 AFAU/kg fabric, especially 100-500 AFAU/kg fabric or 1-3,000 AFAU/L treating solution, preferably 10-1,000 AFAU/L treating solution, especially 100-500 AFAU/L treating solution.

Preferably, the alpha-amylase is the hybrid alpha-amylase shown in SEQ ID NO: 48 comprising a catalytic domain (CD) from Rhizomucor pusilus alpha-amylase having a carbohydrate-binding domain (CBD) from the A. niger.

Normally there are three types of pectic enzymes, pectesterase, depolymerising enzymes, and protopectinase. Preferably, said acid pectinase is an acid pectate lyase, an acid pectin lyase, an acid polygalacturonase, and/or an acid polygalacturonate lyase. More preferably, the acid pectinase is Pectinex BEE XXL, Pectinex Ultra, Pectinex Yield Mash, Pectinex XXL, Pectinex Smash XXL or mixtures thereof.

Preferably, the acid pectinase is from the genus Aspergillus.

Preferably, the acid pectinase can be added into the solution before, simultaneous, or after the addition of acid amylase.

Preferably, the process is carried out at a temperature in the range from 5-90° C., in particular 20 to 90>C. More preferably, the process is carried out at a temperature between 25 and 60° C. for a suitable period of time, preferably between 2 and 24 hours.

Preferably, the pH is in the range between pH 2 to 4.

Preferably, the fabric is made from fibers of natural or man-made origin, cotton fabric, denim, linen, ramie, viscose, lyocell, or cellulose acetate.

Preferably, the fabric is made of fibers from animal origin, in particular silk or wool.

Preferably, the fabric is made of polyester fibers of man-made or natural origin, such as poly(ethylene terephthalate) or poly(lactic acid) or fibers of nylon, acrylic, or polyurethane. The fabric preferably is a polyester containing fabric or garment consists of essentially 100% polyester. The polyester fabric is a polyester blend, such as a polyester and cellulosic blend, including polyester and cotton blends; a polyester and wool blend; a polyester and silk blend, a polyester and acrylic blend; a polyester and nylon blend, a polyester, nylon and polyurethane blend, a polyester and polyurethane blend, rayon (viscose), cellulose acetate and tencel.

In another aspect, the present invention relates to a composition comprising an acid amylase and an acid scouring enzyme. The acid amylase is preferably derived from Aspergillus niger or Rhizomucor pusillus or mixtures thereof. The scouring enzyme is preferably selected from the group consisting of acid cellulase, acid pectinase, acid lipase, acid xylanase and/or acid protease, and mixtures thereof.

Preferably, said acid pectinase is Pectinex® BE XXL, Pectinex® BE Colour, Pectinex® Ultra; Pectinex™ Ultra SP-L, Pectinex® Yield Mash, Pectinex® XXL, Pectinex® Smash XXL, Pectinex® Smash and/or Pectinex™ AR. Said acid pectinase is preferably derived from a strain of Aspergillus. The composition further comprises stabilizer, surfactant, wetting agent, dispersing agents, sequestering agents and emulsifying agents, or a mixture thereof.

In the third aspect, the present invention relates to the use of the composition as described above for simultaneous desizing and scouring.

The present inventors have found that when carrying out a simultaneously desizing and bioscouring process of the invention, as defined in the claims, no demineralization is needed. The demineralization takes place simultaneously and/or after the desizing and the bioscouring of the sized fabric in the same treating solution. Compared to traditional processes involving an acid desizing step and a demineralization step a pH adjusting step is avoided. Another advantage of the invention is that process time is saved/reduced as desizing. bioscouring and demineralization may be carried out simultaneously. Even if the combined desizing and bioscouring and demineralization are not carried out as a one step process, i.e., simultaneously, costs of, e.g., acids and manpower for adding acid(s) are saved/reduced as the pH adjustment step between the traditional acid desizing step and the demineralization step is avoided. As compared to simultaneous desizing and bioscouring under alkaline conditions, simultaneous desizing and bioscouring under acid conditions can remove the demineralization at the same time without additional demineralising procedure.

In the context of the invention, the term “treatment” means the combination of enzymes that provide facilitated processing, such as combined desizing and scouring, combined desizing and biopolishing, combined desizing and abrasion; etc.

In the context of the invention. the term “biopolishing” is a specific treatment of the yarn surface which improves fabric quality with respect to handle and appearance without loss of fabric wettability. The most important effects of biopolishing can be characterised by less fuzz and pilling, increased gloss/luster, improved fabric handle, increased durable softness and improved water absorbency.

In context of the invention, the term “combined” or “combination” means that the combined process steps, or the combination is carried out sequentially or simultaneously in one bath (i.e., same treating solution). In a preferred embodiment the combined process or the combination is carried out simultaneously in one bath (i.e., same treating solution).

In context of the invention the term “fabric” is used interchangeable with the term “textile” and means, in contrast to “used” laundry fabric, newly manufactured, preferably undyed, fabrics, garments, fibres, yarns or other types of processed fabrics. Fabrics can be constructed from fibers by weaving, knitting or non-woven operations. Weaving and knitting require yarn as the input whereas the non-woven fabric is the result of random bonding of fibers (paper can be thought of as non-woven).

Woven fabric is constructed by weaving “filling” or weft yarns between warp yarns stretched in the longitudinal direction on the loom. The wrap yarns must be sized before weaving in order to lubricate and protect them from abrasion at the high speed insertion of the filling yarns during weaving. The filling yarn can be woven through the warp yarns in a “over one—under the next” fashion (plain weave) or by “over one—under two” (twill) or any other myriad of permutations. Strength, texture and pattern are related not only to the type/quality of the yarn but also the type of weave. Generally, dresses, shirts, pants, sheeting's, towels, draperies, etc. are produced from woven fabric.

Knitting is forming a fabric by joining together interlocking loops of yarn. As opposed to weaving, which is constructed from two types of yam and has many “ends”, knitted fabric is produced from a single continuous strand of yarn. As with weaving, there are many different ways to loop yarn together and the final fabric properties are dependent both upon the yarn and the type of knit. Underwear, sweaters, socks, sport shirts, sweat shirts, etc. are derived from knit fabrics.

Non-woven fabrics are sheets of fabric made by bonding and/or interlocking fibers and filaments by mechanical, thermal, chemical or solvent mediated processes. The resultant fabric can be in the form of web-like structures, laminates or films. Typical examples are disposable baby diapers, towels, wipes, surgical gowns, fibers for the “environmental friendly” fashion, filter media, bedding, roofing materials, backing for two-dimensional fabrics and many others.

According to the invention, the process may be applied to any sized fabric known in the art (woven, knitted, or non-woven). The process is applied to newly manufactured sized fabric, as opposed to used and/or soiled fabric to be cleaned during laundry washing. In an embodiment the fabric is made of fibres of natural and/or man-made origin. In another embodiment the fabric is made of fibres from animal origin. In particular, the process of the invention may be applied to cellulose-containing or cellulosic fabrics, such as cotton, viscose, rayon, ramie, linen, cellulose acetate, denim, lyocell (Tencel™, e.g., produced by Courtaulds Fibers), or mixtures thereof, or mixtures of any of these fibers together with synthetic fibres (e.g. polyester, polyamide, acrylic, or polyurethane, nylon, poly(ethylene terephthalate) or poly(lactic acid) or other natural fibers, such as wool and silk., such as viscose/cotton blends, lyocell/cotton blends, viscose/wool blends, lyocell/wool blends, cotton/wool blends; flax (linen), ramie and other fabrics based on cellulose fibers, including all blends of cellulosic fibers with other fibers such as wool, polyamide, acrylic and polyester fibers, e.g., viscose/cotton/polyester blends, wool/cotton/polyester blends, flax/cotton blends etc. The process may also be used on synthetic fabric, e.g., consisting of essentially 100% polyester, polyamide, nylon, respectively. The term “wool,” means any commercially useful animal hair product, for example, wool from sheep, camel, rabbit, goat, lama, and known as merino wool. Shetland wool, cashmere wool, alpaca wool, mohair, etc. and includes woos fiber and animal hair. The process of the invention can be used with wool or animal hair material in the form of top, fiber, yarn, or woven or knitted fabric.

The alpha-amylase used in accordance with the process of the invention may be any acid alpha-amylase, but is preferably of either bacterial or fungal origin.

Preferably the acid alpha-amylase is derived from a filamentous fungus, especially a strain of Aspergillus, Rhizomucor or Meripilus.

The term “acid alpha-amylase” means an alpha-amylase (E.C. 3.2.1.1) which has an optimum activity at a pH in the range of 1 to 7, preferably from 1 to 5 at a temperature of 50° C.

The term “desizing” is intended to be understood in a conventional manner, i.e. the degradation and/or removal of sizing agents from fabric, such as warp yarns in a woven fabric.

The term “fabric containing starch or starch derivatives” is intended to indicate any type of fabric, in particular woven fabric prepared from a celluose-containing material, containing starch or starch derivatives. The fabric is normally undyed and made of cotton viscose, flax, and the like. The main part of the starch or starch derivatives present on the fabric is normally size with which the yarns, normally warp yarns, have been coated prior to weaving.

The term “carbohydrate-binding module (CBM)”, or as often referred to a “carbohydrate-binding domain (CBD)”, is a polypeptide amino acid sequence which binds preferentially to a poly- or oligosaccharide (carbohydrate), frequently—but not necessarily exclusively—to a water-insoluble (including crystalline) form thereof.

Even if not specifically mentioned in connection with the process of the invention, it is to be understood that the enzyme(s) or agent(s) is(are) used in an “effective amount”. The term “effective amount” means an amount of, e.g., alpha-amylase that is capable of providing the desired effect, i.e., desizing of the fabric, as compared to a fabric which has not been treated with said enzyme(s).

### DETAILED DISCLOSURE OF THE INVENTION

The present invention is directed towards providing a process of desizing a sized fabric during manufacture of especially new fabrics.

The desizing step of the invention is in a preferred embodiment followed by a scouring step, preferable an enzymatic scouring step, preferably with a scouring enzyme such as a pectinase, e.g., a pectate lyase, a lipase, a protease, or combination thereof, and a bleaching step, preferably involving bleaching with hydrogen peroxide and/or a hydrogen peroxide generating agent. Relevant scouring processes are described in U.S. Pat. No. 5,578,489, U.S. Pat. No. 5,912,407, and U.S. Pat. No. 6,630,342. Relevant bleach processes are described in U.S. Pat. No. 5,851,233, U.S. Pat. No. 5,752,980, and U.S. Pat. No. 5,928,380. Relevant combined scouring and bleach processes are described in WO 2003/002810 (Novozymes) and WO 2003/002705 (Novozymes).

According to the present invention, fabric may be desized and demineralized simultaneously in the same aqueous treating solution (i.e., one bath) or subsequently in the same or two separate treating solutions (i.e., one or two baths). In a preferred embodiment the desizing and demineralization are carried out simultaneously in the same treating solution (i.e., one bath). The process of the invention may be carried out using traditional sizing/desizing equipment, e.g., pad systems, J-boxes, jets, jiggers, etc. In general, no additional process equipment is needed.

According to the invention simultaneous desizing and demineralization are carried out by incubating sized fabric in an aqueous treating solution having a pH in the range between 1 and 7 which aqueous treating solution comprises an acid alpha-amylase. In a preferred embodiment the pH during incubation is in the range between 1 and 4, especially between pH 2 and 4.

Woven goods are the prevalent form of fabric construction. The weaving process demands a “sizing” of the warp yarn to protect it from abrasion. Starches, unmodified and modified, polyvinyl alcohol (PVA), carboxy methyl cellulose (CMC), waxes and acrylic binders, and mixtures thereof, are examples of typically used sizing agents. The sizing agent may according to the invention be a starch-based or starch derivative-based sizing agent, but may also contain one or more non-starch or starch derivative-based sizing agents. The sizing agent(s) are in general removed after the weaving process as the first step in preparing the woven goods.

One or more other agents including stabilizers, surfactants, wetting agents, dispersing agent, sequestering agents and emulsifying agents, or mixtures thereof, may be present during a desizing process of the invention. The sized fabric is allowed to incubate in the aqueous treating solution for a sufficiently long period of time to accomplish desizing of the sized fabric. The optimal period is dependent upon the type of processing regime and the temperature and can vary from about −15 minutes to several days, e.g., 48 hours. A process of the invention is preferably carried out at a temperature in the range from 5 to 90° C., in particular 20 to 90° C. dependent on the processing regime.

The processing regime can be either batch or continuous with the fabric being contacted by the aqueous treating stream in open width or rope form.

Continuous operations may use a saturator whereby an approximate equal weight of treating solution per weight of fabric is applied to the fabric, followed by a heated dwell chamber where the chemical reaction takes place. A washing section then prepares the fabric for the next processing step. In order to ensure a high whiteness or a good wettability and resulting dyeability, the desizing enzyme(s) and other agents must be thoroughly removed.

Batch processes may take place in one bath (treating solution) whereby the fabric is contacted with e.g., approximately 8-15 times its weight of aqueous treating solution. After an incubation period, the aqueous treating solution is drained, the fabric is rinsed and the next processing step is initiated. Discontinuous PB-processes (i.e., pad-batch processes) involves a saturator whereby an approximate equal weight of aqueous treating solution per weight of fabric is applied to the fabric, followed by a dwell period, which in the case of CPB-process (i.e., cold pad-batch process) might be one or more days. For instance, a CPB-process may be carried out at between 20-40° C. for 8-24 hours or more at a pH in the range between 1 and 7, preferably at a pH in the range between around 1 and 4, especially between pH 2 and 4. Further, a PB-process may be carried out at between 40-90° C. for 1-6 hours at a pH in the range between around 1 and 7, preferably between around pH 1 and 5. more preferably between 1 and 4, especially between pH 2 and 4.

In one embodiment the desizing process of the invention may be carried out using an effective amount of alpha-amylase, preferably acid alpha-amylase, and an acid such as acetic acid or sulphuric acid or the like.

### Enzymes

### Alpha-Amylases

The alpha-amylase(s) used in the process of the invention may be any alpha-amylase preferably of bacterial or fungal origin. In a preferred embodiment the alpha-amylase is an acid alpha-amylase, such as an alpha-amylase or hybrid alpha-amylase disclosed in WO 2005/003311 which is hereby incorporated by reference.

In a preferred embodiment the alpha-amylase include a carbohydrate-binding module (CBM) as defined in WO 2005/03311 preferably a family 20 CBM as defined in WO 2005/003311.

Specifically contemplated are CBMs include the ones selected from the group consisting of Aspergillus kawachii disclosed in SEQ ID NO: 2; Bacillus flavothermus disclosed in SEQ ID NO: 5; Bacillus sp. disclosed in SEQ ID NO: 6; Alcaliphilic Bacillus disclosed in SEQ ID NO: 7; Hormoconis resinae disclosed in SEQ ID NO,: 8: Lentinula edodes disclosed in SEQ ID NO, 9; Neurospora crassa disclosed in SEQ ID NO, 10; Talaromyces byssochlamydiodes disclosed in SEQ ID NO: 11; Geosmithia cylindrospora disclosed in SEQ ID NO: 12; Scorias spodiosa disclosed in SEQ ID NO: 13; Eupenicillium ludwigii disclosed in SEQ ID NO: 14; Aspergillus japonicus disclosed in SEQ ID NO: 15; Penicillium cf. miczynskii disclosed in SEQ ID NO: 16; Mz1 Penicillium sp. disclosed in SEQ ID NO: 17; Thysanospora sp. disclosed in SEQ ID NO: 18; Humicola grisea var thermoidea disclosed in SEQ ID NO: 19; Aspergillus niger disclosed in SEQ ID NO: 20; or Althea rolfsii disclosed in SEQ ID NO: 21.

### Fungal Alpha-Amylases

In an embodiment the fungal alpha-amylase is of yeast or filamentous fungus origin. In a preferred embodiment the fungal alpha-amylase is an acid alpha-amylase.

Preferred alpha-amylases include, for example. alpha-amylases obtainable from Aspergillus species, in particular from Aspergillus niger, A. oryzae, and A. awamori, A. kawachii, such as the acid alpha-amylase disclosed as SWISSPROT P562714 or described in more detail in WO 89/01969 (Example 3). The mature acid alpha-amylase has the amino acid sequence shown as 22-511 of SEQ ID NO: 4, encoded by the DNA sequence shown in SEQ ID NO: 3, or the amino acid sequence shown in SEQ ID NO: 38. Also preferred are alpha-amylase sequences having more than 50%. such as more than 60%, more than 70%, more than 80% or more than 90%, more than 95%, more than 96%, more than 97%, more than 98%, or even more than 99% identity to the amino acid sequence shown in SEQ ID NOS: 4 or 384 respectively.

In another preferred embodiment the alpha-amylase sequence is derived from an A. oryzae acid alpha-amylase. More preferably the alpha-amylase sequence has more than 50%, such as more than 60%, more than 70%, more than 80% or more than 90%, more than 95%, more than 96%, more than 97%, more than 98%, or more than 99% identity to the amino acid sequence shown in SEQ ID NO: 39.

In one embodiment the alpha-amylase is the Aspergillus kawachii alpha-amylase disclosed in SEQ ID NO: 37 which in wildtype form contains a carbohydrate-binding domain (CBD) also shown in SEQ ID NO: 2.

In a preferred embodiment the alpha-amylase is an alpha-amylase having more than 50%, such as more than 60%, more than 70%, more than 80% or more than 90%, more than 95%, more than 96%, more than 97%, more than 98%. or even more than 99% identity to the amino acid sequence shown in SEQ ID NOS: 43, 44, 46 or 47, respectively.

The alpha-amylase may be present in a concentration of 1-3,000 AFAU/kg fabric, preferably 10-1,000 AFAU/kg fabric, especially 100-500 AFAU/kg fabric or 1-3,000 AFAU/L treating solution, preferably 10-1,000 AFAU/L treating solution, especially 100-500 AFAU/L treating solution.

### Bacterial Alpha-Amylases

In an embodiment the alpha-amylase is of bacterial origin. In a preferred embodiment the bacterial alpha-amylase is an acid alpha-amylase.

The bacterial alpha-amylase is preferably derived from a strain of Bacillus, such as Bacillus licheniformis, Bacillus amyloliquefaciens, Bacillus stearothermophilus, Bacillus subtilis, or other Bacillus sp. such as Bacillus sp. NCIB 12289, NCIB 12512 (WO 95/26397), NCIB 12513 (WO 95/26397), DSM 9375 (WO 95/26397), DSMZ 12648 (WO 00160060), DSMZ 12649 (WO 00/60060), KSM AP1378 (WO 97/00324), KSM K36 or KSM K38 (EP 1,022,334). Preferred are the Bacillus sp. alpha-amylases disclosed in WO 95/26397 as SEQ ID NOS. 1 and 2, respectively, the AA560 alpha-amylase disclosed as SEQ ID NO: 2 in WO 00/60060 (i.e. SEQ ID NO: 40 herein), and the #707 alpha-amylase disclosed by Tsukamoto et al., Biochemical and Biophysical Research Communications, 151, pp. 25-31 (1988).

In an embodiment of the invention the bacterial alpha-amylase is the SP722 alpha-amylase disclosed as SEQ ID NO: 2 in WO 95126397 or the AA560 alpha-amylase (SEQ ID NO: 40 herein).

In a preferred embodiment the parent alpha-amylase has one or more deletions in positions or corresponding to the following positions: D183 and G184, preferably wherein said alpha-amylase variant further has a substitution in position or corresponding to position N195F (using the SEQ ID NO: 40 numbering).

In another preferred embodiment the parent alpha-amylase has one or more of the following deletions/substitutions or corresponding to the following deletions/substitutions: Delta (R81−G182); Delta (D183−G184); Delta (D183−G184)+N195F; R181Q+N445Q+K446N; Delta (D183−G184)+R181Q, Delta (D183−G184) and one or more of the following substitutions or corresponding to: R1118K, N195F, R320K, R458K, especially wherein the variant has the following mutations: Δ(D183+G184)+R118K+N195F+R320K+R458K (using the SEQ ID NO: 40 numbering).

In another preferred embodiment the alpha-amylase is the AA560 alpha-amylase shown in SEQ ID NO, 40 further comprising one or more of the following substitutions M9L, M202L, V214T, M323T, M382Y, E345R or the A560 alpha-amylase with all of the following substitutions, M9L, M202L, V214T, M323T, M382Y or M9L, M202L, V214T, M323T and E345R.

Commercially available alpha-amylase products or products comprising alpha-amylases include product sold under the following tradenames: NATALASE™, STAINZYME™ (Novozymes A/S), Bioamylase—D(G), BIOAMYLASE™ L (Biocon India Ltd.), KEMZYM™ AT 9000 (Biozym Ges. m.b.H. Austria). PURASTAR™ ST, PURASTAR™ HPAML, PURAFECT™ OxAm, RAPIDASE™ TEX (Genencor Int. Inc, USA), KAM (Kao, Japan).

The alpha-amylase may be present in a concentration of from about 0.05-150 KNU/L treating solution, preferably 1-100 KNU/L treating solution, especially 2-20 KNU/L treating solution or 0.05-150 KNU/Kg fabric, preferably, 1-100 KNU/kg fabric, especially 2-20 KNU/kg fabric.

### Hybrid Enzyme

The alpha-amylase may in a preferred embodiment be an alpha-amylase comprising a carbohydrate-binding domain (CBD). Such alpha-amylase with a CBD may be a wild type enzyme (see e.g., Aspergillus kawachii above) or a hybrid enzyme (fusion protein) as will be described further below. Hybrid enzymes or a genetically modified wild type enzymes as referred to herein include species comprising an amino acid sequence of an alpha-amylase enzyme (EC 3.2.1.1) linked (i.e., covalently bound) to an amino acid sequence comprising a carbohydrate-binding domain (CBD).

CBD-containing hybrid enzymes, as well as detailed descriptions of the preparation and purification thereof, are known in the art [see, e.g., WO 90/00609, WO 94/24158 and WO 95/16782, as well as Greenwood et al., Biotechnology and Bioengineering, 1994, 44: 1295-1305]. They may, e.g., be prepared by transforming into a host cell a DNA construct comprising at least a fragment of DNA encoding the carbohydrate-binding domain ligated, with or without a linker, to a DNA sequence encoding the enzyme of interest, and growing the transformed host cell to express the fused gene. The resulting recombinant product (hybrid enzyme)—often referred to in the art as a “fusion protein—may be described by the following general formula:

A-CBD-MR-X

In the latter formula, A-CBD is the N-terminal or the C-terminal region of an amino acid sequence comprising at least the carbohydrate-binding domain (CBD) per se. MR is the middle region (the “linker”), and X is the sequence of amino acid residues of a polypeptide encoded by a DNA sequence encoding the enzyme (or other protein) to which the CBD is to be linked.

The moiety A may either be absent (such that A-CBD is a CBD per se, i.e., comprises no amino acid residues other than those constituting the CBD) or may be a sequence of one or more amino acid residues (functioning as a terminal extension of the CBD per se). The linker (MR) may be a bond, or a short linking group comprising from about 2 to about 100 carbon atoms, in particular of from 2 to 40 carbon atoms. However, MR is preferably a sequence of from about 2 to about 100 amino acid residues. more preferably of from 2 to 40 amino acid residues, such as from 2 to 15 amino acid residues.

The moiety X may constitute either the N-terminal or the C-terminal region of the overall hybrid enzyme.

It will thus be apparent from the above that the CBD in a hybrid enzyme of the type in question may be positioned C-terminally, N-terminally or internally in the hybrid enzyme.

### Linker Sequence

The tinker sequence may be any suitable linker sequence. in preferred embodiments the linker sequence is derived from the Athelia rolfsii glucoamylase, the A. niger glucoamylase, the A. kawachii alpha-amylase such as a linker sequence selected from the group consisting of A. niger glucoamylase linker: TGGTTTTATPTGSGSVTSTSKTTATASKTSTSTSSTSA (SEQ ID NO: 22) A. kawachii alpha-amylase linker: T T T T T T A A A T S T S K A T T S S S S S S A A A T T S S S (SEQ ID NO: 23), Athelia rolfsii glucoamylase linker: G A T S P G G S S G S (SEQ ID NO:. 24), and the PEPT linker: P E P T P E P T (SEQ ID NO: 25). In another preferred embodiment the hybrid enzymes has a linker sequence which differs from the amino acid sequences shown in SEQ ID NO: 22, SEQ ID NO: 23, SEQ ID NO: 24, or SEQ ID NO: 25 in no more than 10 positions, no more than 9 positions, no more than 8 positions, no more than 7 positions, no more than 6 positions, no more than 5 positions, no more than 4 positions, no more than 3 positions, no more than 2 positions, or even no more than 1 position.

### Carbohydrate-Binding Domain

A carbohydrate-binding domains (CBD), or as often referred to, a carbohydrate-binding modules (CBM), is a polypeptide amino acid sequence which binds preferentially to a poly- or oligosaccharide (carbohydrate), frequently—but not necessarily exclusively—to a water-insoluble (including crystalline) form thereof.

CBDs derived from starch degrading enzymes are often referred to as starch-binding domains (SBD) or starch-binding modules (SBM). SBDs are CBDs which may occur in certain amylolytic enzymes, such as certain glucoamylases, or in enzymes such as cyclodextrin glucanotransferases, or in alpha-amylases. Likewise, other sub-classes of CBDs would embrace, e.g., cellulose-binding domains (CBDs from cellulolytic enzymes), chitin-binding domains (CODs which typically occur in chitinases), xylan-binding domains (CODs which typically occur in xylanases), mannan-binding domains (CODs which typically occur in mannanases).

CBDs are found as integral parts of large polypeptides or proteins consisting of two or more polypeptide amino acid sequence regions, especially in hydrolytic enzymes (hydrolases) which typically comprise a catalytic domain containing the active site for substrate hydrolysis and a carbohydrate-binding domain (COD) for binding to the carbohydrate substrate in question. Such enzymes can comprise more than one catalytic domain and one, two or three CBDs, and optionally further comprise one or more polypeptide amino acid sequence regions linking the CBD(s) with the catalytic domain(s), a region of the latter type usually being denoted a “linker”. Examples of hydrolytic enzymes comprising a CBD—some of which have already been mentioned above—are cellulases. xylanases, mannanases, arabinofuranosidases, acetylesterases and chitinases. CODs have also been found in algae, e.g., in the red alga Porphyra purpurea in the form of a non-hydrolytic polysaccharide-binding protein.

In proteins/polypeptides in which CBDs occur (e.g., enzymes, typically hydrolytic enzymes), a CBD may be located at the N or C terminus or at an internal position.

That part of a polypeptide or protein (e.g., hydrolytic enzyme) which constitutes a CBD per se typically consists of more than about 30 and less than about 250 amino acid residues.

The “Carbohydrate-Binding Module of Family 20” or a CBM-20 module is in the context of this invention defined as a sequence of approximately 100 amino acids having at least 45% homology to the Carbohydrate-Binding Module (CBM) of the polypeptide disclosed in FIG. 1 by Joergensen et al (1997) in Biotechnol. Lett. 19:1027-1031. The CBM comprises the last 102 amino acids of the polypeptide, i.e., the subsequence from amino acid 582 to amino acid 683. The numbering of Glycoside Hydrolase Families applied in this disclosure follows the concept of Coutinho, P. M. & Henrissat, B. (1999) CAZy—Carbohydrate-Active Enzymes server at URL: http://afmb.cnrs˜mrs.fr/˜cazy/CAZY/index.html or alternatively Coutinho, P. M. & Henrissat, B. 1999; The modular structure of cellulases and other carbohydrate-active enzymes; an integrated database approach. In “Genetics, Biochemistry and Ecology of Cellulose Degradation”, K. Ohmiya, K. Hayashi, K. Sakka, Y. Kobayashi, S. Karita and T. Kimura eds., Uni Publishers Co., Tokyo, pp. 15-23, and Bourne, Y. & Henrissat, B. 2001; Glycoside hydrolases and glycosyltransferases: families and functional modules, Current Opinion in Structural Biology 11:593-600.

Examples of enzymes which comprise a CBD suitable for use in the context of the invention are alpha-amylases, maltogenic alpha-amylases, cellulases, xylanases, mannanases, arabinofuranosidases, acetylesterases and chitinases. Further CBDs of interest in relation to the present invention include CBDs derived from glucoamylases (EC 3.2.1.3) or from CGTases (EC 2.4.1.19).

CBDs derived from fungal, bacterial or plant sources will generally be suitable for use in the context of the invention. Preferred are CBDs of fungal origin, more preferably from Aspergillus sp., Bacillus sp., Klebsiella sp., or Rhizopus sp. In this connection, techniques suitable for isolating the relevant genes are well known in the art.

Preferred for the invention is CBDs of Carbohydrate-Binding Module Family 20. CBDs of Carbohydrate-Binding Module Family 20 suitable for the invention may be derived from glucoamylases of Aspergillus awamori (SWISSPROT Q12537). Aspergillus kawachii (SWISSPROT P23176), Aspergillus niger (SWISSPROT PO₄₀₆₄), Aspergillus oryzae (SWISSPROT P36914), from alpha-amylases of Aspergillus kawachii (EMBL:#AB008370) Aspergillus nidulans (NCBI AAF17100.1), from beta-amylases of Bacillus cereus (SWISSPROT P36924), or from CGTases of Bacillus circulans (SWISSPROT P43379). Preferred is a CBD from the alpha-amylase of Aspergillus kawachii (EMBL:#AB008370) as well as CBDs having at least 50%, 60%. 70%, 80% or even at least 90%, 95%, 96%, 97%, 98%, or 99% identity with the CBD of the alpha-amylase of Aspergillus kawachii (EMBL:#AB008370), i.e., a CBD having at least 50%, 60%, 70%, 80% or even at least 90%, 95%, 96%, 97%, 98%, or 99% identity with the amino acid sequence of SEQ ID NO: 2. Also preferred for the invention are the CBDs of Carbohydrate-Binding Module Family 20 having the amino acid sequences shown in SEQ ID NO, 5, SEQ ID NO, 6, and SEQ ID NO: 7 and disclosed in PCT application no. PCT/DK2004/000456 (or Danish patent application PA 2003 00949) as SEQ ID NO: 1, SEQ ID NO: 2 and SEQ ID NO, 3 respectively. Further 3.0) preferred CBDs include the CBDs of the glucoamylase from Hormoconis sp. such as from Hormoconis resinae (Syn. Creosote fungus or Amorphotheca resinae) such as the CBD in SWISSPROT:Q03045 (SEQ ID NO: 8), from Lentinula sp. such as from Lentinula edodes (shiitake mushroom) such as the OBD of SPTREMBL:Q9P4C5 (SEQ ID NO: 9), from Neurospora sp. such as from Neurospora crassa such as the CBD of SWISSPROT:P14804 (SEQ ID NO: 10), from Talaromyces sp. such as from Talaromyces byssochlamydioides such as the CBD from NN005220 (SEQ ID NO:311), from Geosmithia sp. such as from Geosmithia cylindrospora, such as the CBD of NN48286 (SEQ ID NO: 12)., from Scorias sp. such as from Scorias spongiosa such as the CBD of NN007096 (SEQ ID NO: 13), from Eupenicillium sp. such as from Eupenicillium ludwigii such as the CBD of NN005968 (SEQ ID NO: 14). from Aspergillus sp. such as from Aspergillus japonicus such as the CBD from NN001136 (SEQ ID NO: 15), from Penicillium sp, such as from Penicillium cf. miczynskii such as the CBD of NN48691 (SEQ ID NO: 16), from Mz1 Penicillium sp. such as the CBD of NN48690 (SEQ ID NO: 17), from Thysanophora sp. such as the CBD of NN48711 (SEQ ID NO: 18), and from Humicola sp. such as from Humicola grisea var. thermoidea such as the CBD of SPTREMBL.:Q12623 (SEQ ID NO: 19). Most preferred CBDs include the CBDs of the glucoamylase from Aspergillus sp. such as from Aspergillus niger; such as SEQ ID NO: 20, and Athelia sp. such as from Athelia rolfsii, such as SEQ ID NO: 21. Also preferred according to the invention are any CBD having at least 50%, 60%. 70%, 80% or even at least 90%, 95%, 96%, 97%, 98%, or 99% identity to any of the afore mentioned CBD amino acid sequences.

Further suitable CBDs of Carbohydrate-Binding Module Family 20 may be found at URL: http://afmb.cnrs-mrs.fr/˜cazy/CAZY/index.html).

Once a nucleotide sequence encoding the substrate-binding (carbohydrate-binding) region has been identified, either as cDNA or chromosomal DNA, it may then be manipulated in a variety of ways to fuse it to a DNA sequence encoding the enzyme of interest. The DNA fragment encoding the carbohydrate-binding amino acid sequence and the DNA encoding the enzyme of interest are then ligated with or without a linker. The resulting ligated DNA may then be manipulated in a variety of ways to achieve expression.

In an embodiment the alpha-amylase comprised in the hybrid is an alpha-amylase described above in the “Alpha-amylase”-section. in a preferred embodiment the alpha-amylase is of fungal origin. In a more preferred embodiment the alpha-amylase is an acid alpha-amylase.

In a preferred embodiment the carbohydrate-binding domain and/or linker sequence is of fungal origin. The carbohydrate-binding domain may be derived from an alpha-amylase, but may also be derived from of proteins, e.g., enzymes having glucoamylase activity.

In an embodiment the alpha-amylase is derived from a strain of Aspergillus, or Athelia. In an embodiment the alpha-amylase is derived from a strain of Aspergillus oryzae or Aspergillus niger. In a specific embodiment the alpha-amylase is the A. oryzae acid alpha-amylase disclosed in SEQ ID NO: 39. In a specific embodiment the linker sequence may be derived from a strain of Aspergillus, such as the A. kawachii alpha-amylase (SEQ ID NO: 23) or the A. rolfsii glucoamylase (SEQ ID NO: 24). In an embodiment the C0D is derived from a strain of Aspergillus or Athelia. In a specific embodiment the CBD is the A. kawachii alpha-amylase shown in SEQ ID NO: 1 or the A. rolfsii glucoamylase shown in SEQ ID NO: 21.

Preferred is the embodiment wherein the hybrid enzyme comprises an alpha-amylase sequence derived from the A. niger acid alpha-amylase catalytic domain having the sequence shown in SEQ ID NO: 38 and/or a linker sequence derived from the A. kawachii alpha-amylase shown in SEQ ID NO: 23 or the A. rolfsii glucoamylase shown in SEQ ID NO: 24, and/or the CBD is derived from the A. kawachii alpha-amylase shown in SEQ ID NO: 2, the A. rolfsii glucoamylase shown in SEQ ID NO: 21 or the A. niger glucoamylase shown in SEQ ID NO, 22.

In a preferred embodiment the hybrid enzyme comprises the A. niger acid alpha-amylase catalytic domain having the sequence shown in SEQ ID NO: 38, the A. kawachii alpha-amylase linker shown in SEQ ID NO: 23, and A. kawachii alpha-amylase CBD shown in SEQ ID NO., 2.

In a specific embodiment the hybrid enzyme is the mature part of the amino acid sequence shown in SEQ ID NO: 28 (A. niger acid alpha-amylase catalytic domain-A. kawachii alpha-amylase linker-A. niger glucoamylase CBD), SEQ ID NO: 30 (A. niger acid alpha-amylase catalytic domain-A. kawachii alpha-amylase linker-A. rolfsii glucoamylase CBD), or SEQ ID NO: 32 (A. oryzae acid alpha-amylase catalytic domain-A. kawachii alpha-amylase linker-A. kawachii alpha-amylase CBD), or SEQ ID NO: 34 (A. niger acid alpha-amylase catalytic domain-A. rolfsii glucoamylase linker-A. rolfsii glucoamylase CBD), or SEQ ID NO: 36 (A. oryzae add alpha-amylase catalytic domain-A. rolfsii glucoamylase linker-A. rolfsii glucoamylase CBD) or the hybrid consisting of A. niger acid alpha-amylase catalytic domain (SEQ ID NO: 4 or 384 respectively)-A. kawachii glucoamylase linker (SEQ ID NO: 23)-A. kawachii glucoamylase CBD (SEQ ID NO: 2) or a hybrid enzyme that has an amino acid sequence having at least 50%, 60%, 70%, 80% or even at least 90%, 95%, 96%, 97%, 98%, or 99% identity to any of the afore mentioned amino acid sequences.

In another preferred embodiment the hybrid enzyme has an amino acid sequence which differs from the amino acid sequence amino acid sequence shown in SEQ ID NO: 28 (A. niger acid alpha-amylase catalytic domain-A. kawachii alpha-amylase linker-A. niger glucoamylase CBD), SEQ ID NO: 30 (A. niger acid alpha-amylase catalytic domain-A. kawachii alpha-amylase linker-A. rolfsii glucoamylase CBD), SEQ ID NO: 32 (A. oryzae acid alpha-amylase catalytic domain-A. kawachii alpha-amylase linker-A. kawachii alpha-amylase CBD), SEQ ID NO: 34 (A. niger acid alpha-amylase catalytic domain-A. rolfsii glucoamylase linker-A. rolfsii glucoamylase CBD) or SEQ ID NO: 36 (A. oryzae acid alpha-amylase catalytic domain-A. rolfsii glucoamylase linker-A. rolfsii glucoamylase CBD) or the hybrid consisting of A. niger acid alpha-amylase catalytic domain (SEQ ID NOS: 4 or 38, respectively)-A. kawachii glucoamylase linker (SEQ ID NO: 23)-A. kawachi glucoamylase CBD (SEQ ID NO: 2) in no more than 10 positions, no more than 9 positions, no more than 8 positions, no more than 7 positions, no more than 6 positions, no more than 5 positions, no more than 4 positions, no more than 3 positions, no more than 2 positions. or even no more than 1 position.

Preferably the hybrid enzyme comprises a CBD sequence having at least 50%, 60%, 70%, 80% or even at least 90%, 95%, 96%, 97%, 98%, or 99% identity to any of the amino acid sequences shown in SEQ ID NO: 5, SEQ ID NO: 6. SEQ ID NO: 7, SEQ ID NO, 8, SEQ ID NO: 9. SEQ ID NO: 10, SEQ ID NO: 11, SEQ ID NO, 12. SEQ ID NO: 13, SEQ ID NO: 14, SEQ ID NO-15, SEQ ID NO: 16, SEQ ID NO: 17, SEQ ID NO: 18. SEQ ID NO: 19, SEQ ID NO: 20 or SEQ ID NO: 21. Even more preferred the hybrid enzyme comprises a CBD sequence having an amino acid sequence shown in SEQ ID NO: 5, SEQ ID NO: 6, SEQ ID NO: 7, SEQ ID NO: 84 SEQ ID NO: 94 SEQ ID NO: 10, SEQ ID NO: 11, SEQ ID NO: 12, SEQ ID NO: 13, SEQ ID NO: 14, SEQ ID NO: 15, SEQ ID NO: 18, SEQ ID NO: 19, SEQ ID NO: 20 or SEQ ID NO: 21. In yet another preferred embodiment the CBD sequence has an amino acid sequence which differs from the amino acid sequence amino acid sequence shown in SEQ ID NO: 5, SEQ ID NO: 6, SEQ ID NO: 7, SEQ ID NO: 8. SEQ ID NO: 94 SEQ ID NO: 10, SEQ ID NO-11, SEQ ID NO: 12, SEQ ID NO: 13, SEQ ID NO: 14, SEQ ID NO: 15., SEQ ID NO: 16, SEQ ID NO: 17, SEQ ID NO; 18, SEQ ID NO: 19, SEQ ID NO: 20 or SEQ ID NO: 21 in no more than 10 amino acid positions, no more than 9 positions, no more than 8 positions, no more than 7 positions, no more than 6 positions. no more than 5 positions, no more than 4 positions, no more than 3 positions, no more than 2 positions, or even no more than 1 position.

In a most preferred embodiment the hybrid enzyme comprises a CBD derived from a glucoamylase from A. rolfsii, such as the glucoamylase from A. rolfsii AHU 9627 disclosed in U.S. Pat. No. 4,727,026.

### Acid Scouring Enzymes

Any acid scouring enzyme may be used according to the invention. The acid scouring enzyme may be an acid enzyme selected from the group consisting of pectinase. cellulase, lipase, protease, xyloglucanase, cutinase and a mixture thereof. A scouring enzyme is “acid” in context of the present invention when the pH optimum under the conditions present during simultaneously desizing and scouring is below 7, such as between 1-7, preferably below 5, such as between 1-5, especially below 4, such as between 1-4.

Various scouring enzymes are known as:

Polygalacturonase (EC 3.2.1.15) catalyzes the random hydrolysis of 1,4-alpha-D-galactosiduronic linkages in pectate and other galacturonans. Examples of other names are: Pectin depolymerase; pectinase: endopolygalacturonase; endo-polygalacturonase; and endogalacturonase. The systematic name is poly(1,4-alpha-D-galacturonide)glycanohydrolase.

Pectin lyase (EC 4.2.2.10) catalyzes the eliminative cleavage of (1,4)-alpha-D-galacturonan methyl ester to give oligosaccharides with 4-deoxy-6-O-methyl-alpha-D-galact-4-enuronosyl groups at their non-reducing ends. Examples of other names are: Pectin trans-eliminase: polymethylgalacturonic transeliminase; and pectin methyltranseliminase. The systematic name is (1,4)-6-O-methyl-alpha-D-galacturonan lyase.

Pectate lyase (EC 4.2.2.2) catalyzes the eliminative cleavage of (1,4)-alpha-D-galacturonan to give oligosaccharides with 4-deoxy-alpha-D-galact-4-enuronosyl groups at their non-reducing ends. Examples of other names are: pectate transeliminase; polygalacturonic transeliminase; and endopectin methyltranseliminase. The systematic -name is (1,4)-alpha-D-galacturonan lyase.

Pectinesterase (EC 3.1.1.11) catalyzes the reaction: pectin+n H₂O=n methanol+pectate. Examples of other names are: Pectin demethoxylase; pectin methylesterase; and pectin methyl esterase. The systematic name is pectin pectylhydrolase.

Pectate dissaccharide-lyase (EC 4.2.2.9) catalyzes the eliminative cleavage of 4-(4-deoxy-alpha-D-galact-4-enuronosyl)-D-galacturonate from the reducing end of pectate, i.e., de-esterified pectin. Examples of other names are: Pectate exo-lyase; exopectic acid transeliminase, exopectate lyase, and exopolygalacturonic acid-trans-eliminase. The systematic name: is (1-4)-alpha-D-galacturonan reducing-end-disaccharide-lyase.

The EC numbering is according to the Recommendations of the Nomenclature Committee of the International Union of Biochemistry and Molecular Biology on the Nomenclature and Classification of Enzyme-Catalysed Reactions published in Enzyme Nomenclature 1992 (Academic Press, San Diego, Calif., with Supplement 1 (1993). Supplement 2 (1994), Supplement 3 (1995). Supplement 4 (1997) and Supplement 5 (in Eur. J. Biochem. 1994, 223: 1-5; Eur. J. Biochem. 1995, 232: 1-6; Eur. J. Biochem. 1996, 237: 1-5; Eur. J. Biochem. 1997, 250: 1-6, and Eur. J. Biochem. 1999, 264: 610-650; respectively).

In a preferred embodiment the acid pectinase is a pectate lyase, a pectin lyase, a polygalacturonase, or a polygalacturonate lyase.

The term “pectinase” is intended to include any acid pectinase enzyme. Pectinases are a group of enzymes that hydrolyse glycosidic linkages of pectic substances mainly poly-1,4-alpha-D-galacturonide and its derivatives (see reference Sakai et al., Pectin, pectinase and propectinase: production, properties and applications, in: Advances in Applied Microbiology, Vol. 39, pp. 213-294 (1993)) which enzyme is understood to include a mature protein or a precursor form thereof, or a functional fragment thereof, which essentially has the activity of the full-length enzyme. Furthermore, the term pectinase enzyme is intended to include homologues or analogues of such enzymes.

Preferably the acid pectinase is an enzyme which catalyzes the random cleavage of alpha-1,4-glycosidic linkages in pectic acid also called polygalacturonic acid by transelimination such as the enzyme class polygalacturonate lyase (EC 4.2.2.2) (PGL) also known as poly(1,4-alpha-D-galacturonide) lyase also known as pectate lyase. Also preferred is a pectinase enzyme which catalyzes the random hydrolysis of alpha-1,4-glycosidic linkages in pectic acid such as the enzyme class polygalacturonase (EC 3.2.1.15) (PG) also known as endo-PG. Also preferred is a pectinase enzyme such as polymethylgalcturonate lyase (EC 4.2.2.10) (PMGL), also known as Endo-PMGL, also known as poly(methyoxygalacturonide)lyase also known as pectin lyase which catalyzes the random cleavage of alpha-1,4-glycosidic linkages of pectin. Other preferred pectinases are galactanases (EC 3.2.1.89), arabinanases (EC 3.2.1.99), pectin esterases (EC 3.1.1.11), and mannanases (EC 3.2.1.78).

For the purposes of the invention, the source of the above enzymes including pectin lyase, pectate lyase and pectinesterase is not critical, e.g., the enzymes may be obtained from a plant, an animal, or a microorganism such as a bacterium or a fungus, e.g., a filamentous fungus or a yeast. The enzymes may, e.g., be obtained from these sources by use of recombinant DNA techniques as is known in the art. The enzymes may be natural or wild-type enzymes, or any mutant, variant, or fragment thereof exhibiting the relevant enzyme activity, as well as synthetic enzymes, such as shuffled enzymes, and consensus enzymes. Such genetically engineered enzymes can be prepared as is generally known in the art. e.g. by site-directed mutagenesis, by PCR (using a PCR fragment containing the desired mutation as one of the primers in the PCR reactions), or by Random Mutagenesis. The preparation of consensus proteins is described in, e.g., EP 897985.

The pectinase may be a component occurring in an enzyme system produced by a given micro-organism, such an enzyme system mostly comprising several different pectinase components including those identified above.

Alternatively, the pectinase may be a single component, i.e., a component essentially free of other pectinase enzymes which may occur in an enzyme system produced by a given micro-organism, the single component typically being a recombinant component, i.e., produced by cloning of a DNA sequence encoding the single component and subsequent cell transformed with the DNA sequence and expressed in a host. Such useful recombinant enzymes., especially pectinase, pectin lyases and polygalacturonases are described in detail in, e.g., WO 93/020193. WO 02/092741, WO03/095638 and WO 2004/092479 (from Novozymes A/S) which are hereby incorporated by reference in their entirety including the sequence listings. The host is preferably a heterologous host, but the host may under certain conditions also be the homologous host.

In a preferred embodiment the pectinase used according to the invention is derived from the genus Aspergillus.

In a still preferred embodiment, the pectinase is the protopectinase having an amino acid sequence of SEQ ID NO: 1 of JP 11682877 or the protopectinase having an amino acid sequence generated by deletion, substitution or insertion of one amino acid or several amino acids in the amino acid sequence and having an activity at the same level as or a higher level than the level of the activity of the protopectinase with the amino acid sequence of SEQ ID NO: 1 of JP 11682877.

The pectinase, such as especially pectate lyase, may preferably be present in a concentration in the range from 1-1,500 APSU/kg fabric, preferably 10-1,200 APSU/kg fabric, especially 100-1,000 APSU/kg fabric.

Commercially available acid pectate lyases according to present invention include Pectinex® BE XXL, Pectinex® BE Colour, Pectinex® Ultra; Pectinex Ultra SP-L, Pectinex® Yield Mash, Pectinex® XXL, Pectinex® Smash XXL, Pectinex® Smash, Pectinex™ M AR from Novozymes A/S, Denmark.

### Proteases

Any protease suitable for use in acid solutions can be used. Suitable proteases include those of animal, vegetable or microbial origin. Microbial origin is preferred. Chemically or genetically modified mutants are included. The protease may be a serine protease, preferably an acid microbial protease or a trypsin-like protease. Examples of acid proteases are subtilisins, especially those derived from Bacillus, preferably Bacillus lentus or Bacillus clausii, e.g., subtilisin Novo, subtilisin Carlsberg, subtilisin 309, subtilisin 147 and subtilisin 168 (described in WO 89/06279).

Preferred commercially available protease enzymes include those sold under the trade names ALCALASE™, SAVINASE™ 16 L Type Ex, PRIMASE™, DURAZYM™, and ESPERASE™ (Novozymes A/S, Denmark), those sold under the tradename OPTICLEAN™, OPTIMASE™, PROPARASE™, PURAFECT™, PURAPECT™ MA and PURAPECT™ OX, PURAFECT™ OX-1 and PURAFECT™ OX-2 by Genencor International Inc., (USA).

In an embodiment of the process of the invention a protease may be present in a concentration from 0.001-10 KNPU/L, preferably 0.1-1 KNPU/L, especially around 0.3 KNPU/L or 0.001-10 KNPU/kg fabric, preferably 0.1-KNPU/kg fabric especially around 0.3 KNPU/kg fabric.

### Lipases

Any lipase suitable for use in acid solutions can be used. Suitable lipases include those of bacterial or fungal origin. Chemically or genetically modified mutants are included.

Examples of useful lipases include a Representative acid lipase enzymes include Lipolase™, Lipolase™, Ultra, Palatase™, A, Palatase™, M and Lipozyme™. commercially available from Novo Industri A/S. These acid lipase enzymes are 1.3-specific lipase enzymes that hydrolyze the fatty acid at the 1 and 3 position of the triglyceride. Another representative acid lipase enzyme is the Yeast Lipase-BCC commercially available from Bio-Cat, Inc. This enzyme is derived from a select strain of Candida cylindracea and is a non-specific lipase enzyme which hydrolyzes the fatty acid at all three positions of the triglyceride.

In an embodiment of the process of the invention a lipase enzyme may be present in a concentration from 0.01-100 LU/L treating solution, preferably 1-10 LU/L treating solution, especially around 1 LU/L treating solution or from 0.01-100 LU/kg fabric, preferably 1-10 LU/kg fabric, especially around 1 LU/kg fabric.

### Cellulases

In the present context, the term “cellulase or “cellulolytic enzyme” refers to an enzyme, which catalyzes the degradation of cellulose to glucose, cellobiose, triose and other cellooligosaccharides. Cellulose is a polymer of glucose linked by beta-1,4-glucosidic bonds. Cellulose chains form numerous intra- and intermolecular hydrogen bonds, which result in the formation of insoluble cellulose microfibrils. Microbial hydrolysis of cellulose to glucose involves the following three major classes of cellulases: endo-1,4-beta-glucanases (EC 3.2.1.4), which cleave beta-1,4-glucosidic links randomly throughout cellulose molecules; cellobiohydrolases (EC 3.2.1.91) (exoglucanases), which digest cellulose from the nonreducing end, and beta-glucosidases (EC 3.2.1.21), which hydrolyse cellobiose and low-molecular-mass cellodextrins to release glucose. Most cellulases consist of a cellulose-binding domain (CBD) and a catalytic domain (CD) separated by a linker rich in proline and hydroxy amino acid residues. In the specification and claims, the term “endoglucanase” is intended to denote enzymes with cellulolytic activity, especially endo-1,4-beta-glucanase activity, which are classified in EC 3.2.1.4 according to the Enzyme Nomenclature (1992) and are capable of catalyzing (endo)hydrolysis of 1,4-beta-D-glucosidic linkages in cellulose. lichenin and cereal beta-D-glucans including 1,4-linkages in beta-D-glucans also containing 1,3-linkages. Any cellulase suitable for use in acid solutions can be used. Suitable cellulases include those of bacterial or fungal origin. Chemically or genetically modified mutants are included. Suitable cellulases are disclosed in U.S. Pat. No. 4,435,307, which discloses fungal cellulases produced from Humicola insolens. Especially suitable cellulases are the cellulases having colour care benefits. Examples of such cellulases are cellulases described in European patent application No. 0 495 257, WO 91/17243 and WO 96/29397.

The acidic cellulase enzyme specific to hydrolysis of the polymeric cellulose produced by Acetobacter bacteria can be derived from certain strains of Trichoderma reesei or Aspergillus niger, or their mutants or variants either naturally or artificially induced. As used herein, Trichoderma reesei denotes microorganisms known by that name, as well as those microorganisms classified under the names Trichoderma longibrachiatum and Trichoderma viride. Any cellulase enzyme or enzyme complex that is specific to hydrolysis of cellulose produced by Acetobacter bacteria can be used.

A representative acid cellulase enzyme is the Cellulase Tr Concentrate multi-enzyme acid cellulase complex, which is commercially available from Solvay Enzymes, Inc. Cellulase Tr Concentrate is a food grade cellulase complex obtained by controlled fermentation of a selected strain of Trichoderma reesei. This enzyme complex consists of both exoglucanases and endoglucanases that directly attack native cellulose, native cellulose derivatives, and soluble cellulose derivatives. This enzyme complex specifically hydrolyzes the beta-D,4-glucosidic bonds of bacterial cellulose, in particular the polymeric bacterial cellulose produced by Acetobacter bacteria, as well as its oligomers and derivatives (U.S. Pat. No. 5,975,095).

Another representative cellulase enzyme commercially available from Solvay Enzymes, Inc. is Cellulase TRL multi-enzyme liquid cellulase complex. Cellulase TRL cellulose enzyme complex is derived from Trichoderma reesei in the same manner as Cellulase Tr Concentrate enzyme complex, but is prepared and sold in liquid form. Its activity against bacterial cellulose has been demonstrated to be equivalent to that of Cellulase Tr Concentrate enzyme complex.

Other suitable enzymes for use in the present invention include Celluzyme Acid P enzyme and Celluclast 1.5 L, both commercially available from Novo Nordisk; Multifect™. Cellulase 300 enzyme, commercially available from Genencor International, and Rapidase®. Acid Cellulase enzyme, commercially available from Gist-Brocades B. V. Still other cellulase enzymes or cellulase enzyme complexes are suitable for use in the present invention, provided they exhibit specific hydrolytic activity directed at the beta-glucosidic linkage characteristic of the polymeric bacterial cellulose produced by microorganisms such as Acetobacter bacteria (U.S. Pat. No. 5,975,095).

In an embodiment of the process of the invention the cellulase may be used in a concentration in the range from 0.001-10 g enzyme protein/L treating solution preferably 0.005-5g enzyme protein/L treating solution, especially 0.01-3g enzyme protein/L solution or from 0.001-10g enzyme protein/kg fabric, preferably 0.005-5 g enzyme protein/kg fabric especially 0, 01-3g enzyme protein/kg fabric. In an embodiment the cellulose is used in a concentration of from 0.1-1,000 ECU/g fabric, preferably 0.52000 ECU/g fabric, especially 1-500 ECU/g fabric.

### Cutinase

A cutinase is an enzyme capable of degrading cutin, cf. e.g., Lin T S & Kolattukudy P E, J. Bacteriol. 1978, 133(2): 942-951. Cutinases, for instance, differs from classical lipases in that no measurable activation around the critical micelle concentration (CMC) of the tributyrine substrate is observed. Also, cutinases are considered belonging to a class of serine esterases. The cutinase may also be a cutinase derived from Humicola insolens disclosed in WO 96/13580. The cutinase may be a variant such as one or the variants disclosed in WO 00/34450 and WO 01/92502 which is hereby incorporated by reference.

Examples of cutinases are those derived from Humicola insolens (U.S. Pat. No. 5,827,719); from a strain of Fusarium, e.g., F. roseum culmorum, or particularly F. solani pisi (WO 90/09446; WO 94/14964, WO 94/03578). The cutinase may also be derived from a strain of Rhizoctonia e.g., R. solani, or a strain of Alternaria, e.g., A. brassicicola (WO 94/03578) or variants thereof such as those described in WO 00134450, or WO 01/92502. The cutinase may also be of bacterial origin, such as a strain of Pseudomonas, preferably Pseudomonas mendocina disclosed in WO 01/34899.

The cutinase may be added in a concentration of 0.001-25,000 micrograms enzyme protein/gram fabric, preferably 0.01-10,000 micrograms enzyme protein/g fabric, especially 0.05-1,000 micrograms enzyme protein/g fabric.

### Xyloglucanase

A xyloglucanase is a xyloglucan specific enzyme capable of catalyzing the solubilization of xyloglucan to xyloglucan oligosaccharides. According to IUBMB Enzyme Nomenclature (2003) a xyloglucanase is classified as EC 3.2.1.151. Pauly et al. (Glycobiology, 1999, 9:93-100) disclose a xyloglucan specific endo-beta-1,4-glucanase from Aspergillus aculeatus. A xyloglucanase used according to the invention may be derived from micro-organisms such as fungi or bacteria. Examples of useful xyloglucanases are family 12 xyloglucan hydrolyzing endoglucanases, in particular family 12 xyloglucan hydrolyzing endoglucanases, obtained from, e.g., Aspergillus aculeatus as described in WO 94/14953. Another useful example is a xyioglucanase produced by Trichoderma, especially EGIII. The xyioglucanase may also be derived from a bacterium from the genus Bacillus, including Bacillus licheniformis, Bacillus agaradharens or Bacillus firmus. The xyloglucanase may also be an endoglucanase with xyloglucanase activity and low activity towards insoluble cellulose and high activity towards soluble cellulose, e.g., family 7 endoglucanases obtained from, e.g., Humicola insolens.

The xyloglucanase may be added in a concentration of 0.001-25,000 micrograms enzyme protein/gram fabric. preferably 0.01-10,000 micrograms enzyme protein/g fabric, more preferably 0.05-1.000 micrograms enzyme protein/g fabric. in particular 0.5-500 micrograms enzyme protein/gram fabric.

### Composition of the Invention

In the second aspect the invention relates to a composition suitable for use in the process of the invention. The composition may be a solid or liquid (aqueous) composition and may be a concentrated composition or a ready-to-use composition.

Thus, in this aspect the invention relates to a composition comprising an acid alpha-amylase and an acid scouring enzyme.

The enzymes comprised may preferably be the ones mentioned in the “Enzymes” section above.

In a preferred embodiment the acid alpha-amylase derived from a strain of Bacillus sp., preferably from a strain of B. licheniformis, B. amyloliquefaciens, B. stearothermophilus, Bacillus sp. NCIB 12289, NCIB 12512, NCIB 12513 or DSM 9375, or DSMZ no. 12649, KSM AP1378, or KSM K36 or KSM K38.

The Bacillus alpha-amylase may be a variant having one or more deletions in positions D183 and G184, respectively, and may further have a substitution in position N195F (using SEQ ID NO: 4 numbering). The Bacillus alpha-amylase variant may also be one having one or more deletions in position D183 and G184, and may further have one or more of the following substitutions: R118K, N195F, R320K, R458K (using SEQ ID NO: 6 numbering).

Specifically the Bacillus variant may have a double deletion in positions D183 and G184 and further comprise the following substitutions: R118K+N195F+R320K+R458K (using SEQ ID NO: 6 numbering).

The acid scouring enzyme(s) is(are) selected from the group consisting of acid pectinase, cellulase, lipase, protease, cutinase, xyloglucanase, and mixtures thereof.

In a preferred embodiment the acid pectinase is a pectate lyase, preferably a pectate lyase derived from a strain of Bacillus, preferably a strain of Bacillus licheniformis, Bacillus alcalophllus, Bacillus pseudoalcalophilus, and Bacillus clarkia, especially the species Bacillus licheniformis.

Further agents suitable for the process to be performed may be added separately or be comprised in the composition of the invention. Examples of such agents include stabilizer, surfactant, wetting agent, dispersing agent, sequestering agent and emulsifying agent and mixtures thereof.

Although the acid alpha-amylase and acid scouring enzyme may be added as such, it is preferred that it is formulated into a suitable composition. Thus, the enzymes may be used in the form of a granulate, preferably a non-dusting granulate, a liquid, in particular a stabilized liquid. a slurry, or in a protected form. Dust free granulates may be produced, e.g., as disclosed in U.S. Pat. Nos. 4,106,991 and 4,661,452 (both to Novozymes A/S) and may optionally be coated by methods known in the art.

Liquid enzyme preparations may, for instance, be stabilized by adding a polyol such as, e.g., propylene glycol, a sugar or sugar alcohol or acetic acid, according to established methods. Other enzyme stabilizers are well known in the art. Protected enzymes may be prepared according to the method disclosed in EP 238 216.

In principle the composition of the invention comprising an acid alpha-amylase and a scouring enzyme may contain any other agent to be used in the combined process of the invention.

The composition of the invention comprises in a preferred embodiment at least one further component selected from the group consisting of stabilizers, surfactants, wetting agents, dispersing agents, sequestering agents and emulsifying agents. All of such further components suitable for textile use are well know in the art.

Suitable surfactants include the ones mentioned in the “Detergent” section above. The wetting agent serves to improve the wettability of the fibre whereby a rapid and even desizing and scouring may be obtained. The emulsifying agent serves to emulsify hydrophobic impurities present on the fabric. The dispersing agent serves to prevent that extracted impurities redeposit on the fabric. The sequestering agent serve to remove ions such as Ca, Mg and Fe, which may have a negative impact on the process and preferred examples include caustic soda (sodium hydroxide) and soda ash (sodium carbonate).

### Use of the Composition of the Invention

In the third aspect the invention relates to the use of the composition of the invention in a simultaneous desizing and scouring process, preferably the process of the invention. in a preferred embodiment the composition of the invention is used in a process of the invention.

The invention described and claimed herein is not to intend to limit the scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

### Materials & Methods

### Enzymes

Acid Amylase A: Wild type acid alpha-amylase derived from Aspergillus niger disclosed in SEQ ID NO; 38

Acid Amylase B: Hybrid alpha-amylase shown in SEQ ID NO: 48 comprising a catalytic domain (CD) from Rhizomucor pusillus alpha-amylase having a carbohydrate-binding domain (CBD) from the A. niger.

Acid pectinase A (Pectinex BEE XXL, Novozymes A/S): A pectolytic liquid enzyme preparation produced by Aspergillus species.

Acid pectinase B (Pectinex Ultra; Novozymes A/S): A highly active pectolytic enzyme preparation containing a range of hemicellulolytic activities, produced by a selected strain of Aspergillus aculeatus.

Acid pectinase C (Pectinex Yield Mash, Novozymes A/S)

Acid pectinase D (Pectinex XXL, Novozymes A/S)

Acid pectinase E (Pectinex Smash XXL, Novozymes A/S).

Enzyme classification numbers (EC numbers) referred to in the present specification with claims are in accordance with the Recommendations (1992) of the Nomenclature Committee of the International Union of Biochemist and Molecular Biology, Academic Press Inc, 1992.

### Fabric

460U Interlock Knits (Testfabrics, Inc.)

Vlisco fabric (from Vlisco Helmond B.V.)

### Buffer

### Citrate Buffer

### 1) 10 mM Citrate Buffer (pH 3.0)

1.954 g of Citric acid monohydrate and 0.206 g of Sodium Citrate dihydrate are dissolved in 1 L of de-ionized water.

### 2) 10 mM Citrate Buffer (pH 4.0)

1.376 g of Citric acid monohydrate and 1.015 g of Sodium Citrate dihydrate are dissolved in 1 L of de-ionized water,

### Methods:

### Determination of Homology

For purposes of the present invention, the degree of homology is determined as the degree of identity between two amino acid sequences as determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10, and gap length penalty of 10. Pairwise alignment parameters were Ktuple=1, gap penalty=3, windows=5, and diago-nals=5].

### Acid Alpha-Amylase Activity (AFAU Assay)

When used according to the present invention the activity of any acid alpha-amylase may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase an endo-alpha-amylase (1,4-alpha-D-glucan-glucano-hydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

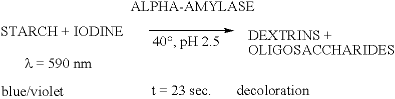

Standard Conditions/Reaction Conditions:

| | |
|---|---|
| Substrate: | Soluble starch, approx, 0.17 g/L |
| Buffer: | Citrate, approx. 0.03 M |
| Iodine (I2): | 0.03 g/L |
| CaCl₂: | 1.85 mM |
| pH: | 2.50 ± 0.05 |
| Incubation temperature: | 40° C. |
| Reaction time: | 23 seconds |
| Wavelength: | 590 nm |
| Enzyme concentration: | 0.025 AFAU/mL |
| Enzyme working range: | 0.01-0.04 AFAU/mL |

A folder EB-SM-0259.02/01 describing this analytical method in more detail is available upon request to Novozymes A/IS Denmark, which folder is hereby included by reference.

### Alpha-Amylase Activity (FAU)

The amylolytic activity may be determined using (4,6-ethylidene(G7)-p-nitrophenyl(G1)-α,-D-maltoheptaoside (ethylidene-G7PNP) as substrate. This method is based on the break-down of ethylidene-G7PNP by the enzyme to glucose and the yellow-colored p-nitrophenol. The rate of formation of p-nitrophenol can be observed by Konelab 30. This is an expression of the reaction rate and thereby the enzyme activity.

The enzyme activity is determined relative to an enzyme standard, 1 FAU is defined as the amount of enzyme which degrades 5.260 mg starch dry matter per hour under the below mentioned standard conditions.

Reaction conditions

| | |
|---|---|
| Temperature | 37° C. |
| pH | 7.15 |
| Substrate concentration | 1.86 mM |
| Wavelength | 405 nm |
| Reaction time | 5 min |
| Measuring time | 2 min |
| Enzyme concentration | 0.46-2.29 mFAU(F)/ml |

A folder EB-SM-0216.02-D describing this analytical method in more detail is available upon request to Novozymes A/S, Denmark, which folder is hereby included by reference.

### Determination of Pectin Transeliminase Activity (UPTE)

The acid pectinase activity may be determined by degrading an Obipectin solution relative to an enzyme standard under the conditions given as below:

Reaction:

| | |
|---|---|
| Substrate concentration | 0.5% Obipectin |
| Temperature | 30° C. |
| pH | 5.4 |
| Reaction time | 10 minutes |
| Absorbance | 238 nm |

One pectin transeliminase unit (UPTE) is defined as the amount of enzyme which raises absorbance by 0.01 absorbance units per minute under standard conditions.

A folder EB-SM-0368.02/01 describing this analytical method in more detail is available upon request to Novozymes A/s, Denmark which folder is hereby included by reference.

### Determination of Polygalacturonase Activity (PGU)

The activity of acid pectinases may be determined by degrading polygalacturonic acid relative to an enzyme standard under the conditions given as below:

Reaction conditions

| | |
|---|---|
| Buffer | Phosphate, 70 mM; Citrate 30 mM |
| Polygalacturonic acid | 19 g/L |
| pH | 3.5 |
| Temperature | 30° C. |
| Time | 30 minutes |
| Polygalacturonase | 400 PGU/L |
| Sample concentration | 9 PGU/mL |

Upon degradation of polygalacturonic acid, the viscosity will reduce, which is proportional to Polygalacturonase activity in the unknown samples.

A folder EB-SM-0615.02 describing this analytical method in more detail is available upon request to Novozymes A/S, Denmark, which folder is hereby included by reference,

### Desizing (Tegewa Method)

The starch size residue is determined visually by comparing an iodine stained fabric swatch to a standard set of photos with 1-9 scale where I is dark blue and 9 has no color stain. The iodine stain solution is made by dissolving 10 g KI in 10 ml water, add 0.635 g 12, and 200 mL ethanol in deionized water to make total 1 L solution. A fabric sample is cut and immersed in the iodine solution for 60 seconds and rinsed in deionized water for about 5 seconds. The fabric sample is rated by at least two professionals after excess water in the sample is pressed out. An average number is given. Method and standard scales obtainable from Verband TEGEWA, Karlstrasse 21, Frankfurt a.M. Germany.

### Pectin Removal

The pectin residue on fabric was determined quantitatively. The principle is that ruthenium red binds to polyanionic compounds like unmethylated pectin. The level of pectin on the fabric is proportional to the concentration of ruthenium red on the cotton fabric which is linearly proportional to Kulbelka-Munk function (i.e., K/S). The color reflectance (R) of ruthenium red stained fabric was measured at 540 nm (Macbeth colorimeter, Model # CE-7000) and automatically calculated into a K/S value by:

K/S=(1−R)²/2R).

The % pectin removal was calculated using the following formula:

%-pectin removal=1−%Res. Pectin=1−100*(K/S−K/S₀)/(K/S₁₀₀−K/S₀)

where K/S₁₀₀ was from fabric with 100% pectin, typically original untreated fabric, while K/S₀ was from the fabric with 0% residual pectin, typically heavily scoured and bleached fabric. Based on information from John H. Luft and described in an article “Ruthenium red and Violet I. Chemistry” 1971, the stain solution was prepared by dissolving 0.2 g/l ruthenium red, 1.0 g/l ammonium chloride, 2.5 ml/128% ammonium hydroxide solution, 1.0 g/l Silwet L-77, and 1.0 g/l Tergitol 15-S-12 in distilled water to make total 1 liter solution. The solution was made daily before use. During staining, 100 mL dye solution was used for 1 gram of fabric. The fabric swatches were incubated in ruthenium red solution for 15 minutes at room temperature. The swatch was rinsed in a strainer and then rinsed in distilled water (100 ml/l gram fabric) at 60° C. for 10 minutes. The color reflectance was measured after dry.

### Fabric Wettability

Fabric wettability was measured using a drop test method according to AATCC test method 79-1995. A drop of water was allowed to fall from a fixed height (1 cm) onto the taut surface of a test specimen. The time required for the specular reflection of the water drop to disappear was measured and recorded as wetting time.

### Wicking Test

The wicking height of textiles is one of the indicators for absorbency. Cut a rectangular fabric swatch 25 cm (warp and weft direction)×4 cm. If the sample is not available in this size to test, adjust the method to fit the sample. Using a waterproof/dye-proof pen, draw a line across the top of the sample 1.5 cm from the top of the swatch and 3 cm from the bottom of the sample. Draw a line across the sample 19 cm from the bottom of the swatch. Attach a paper clamp with a weight to the bottom of the fabric. Place the top of the swatch in the center of the thermometer clamp, so that the line is at the bottom of the clamp. Fill a beaker about half way (at least 5 cm above bottom of glass) with 1 g/L dye solution (e.g., reactive blue). Adjust the clamp with the swatch until the surface of the dye solution is even with the line at the bottom of the fabric. Start the timer as soon as the swatch is in place. Measure the height that the dye solution has wicked up from the surface of the dye solution after 30 min. Remove the swatch and allow it to air dry on a flat surface.

### EXAMPLES

### Example 1

### Scouring Cotton Fabric with Acid Pectinase A

A 100% 460U cotton fabric was purchased from Test Fabrics. Fabric swatches were cut to about 2 g each.

Two buffers were made for this study. Buffer pH 3 was made by dissolving 1.954 g Citric acid monohydrate and 0.206 g sodium citrate dehydrate in 1 liter de-ionized. Buffer pH 4 was made by dissolving 1.3:76g citric acid monohydrate and 1.015 g sodium citrate dehydrate in 1 liter de-ionized. The scouring was conducted with a Lab-O-Mat. The beaker was filled with 40 ml buffer and two pieces of pre-cut fabric,

1. Pre-rinse: The wetting agent, Leophan, was added to the buffer to a concentration of 0.25 g/L. Then the temperature was increased to 400 for pre-rinse. After 10 min, the liquid was drained.
2. Bio-scouring: The beaker with pre-rinsed fabrics was filled with 40 ml buffer. Acid pectinase was added to each beaker as specified. In the meanwhile, the second wetting agent, Keirlon Jet B, was dosed to a concentration of 1 g/L. Temperature was raised to 55° C. and kept for 30 min.
3. Inactivation: After the required time reached, add the Dekol NS in the machine/beaker then raised the temperature to 95° C. and run for 15 min, decreased the temperature to 70° C., drained.
4. Hot rinse: Filled in water and incubated at 70° C. for 10 min
5. Cold rinse: Filled in cold water and rinsed for 10 min
6. Spinned off the water on the fabrics and air dry.
7. Measured residual pectin and wetting time in the treated fabrics.

The result of the test is shown in Table 1.

### Example 2

### Scouring Cotton Fabric with Acid Pectinase B

The same fabric swatch and buffers were prepared as in Example 1. Acid Pectinase B had different enzyme composition compared to Acid Pectinase A. The performance of pectin removal was shown in Table 1. Both enzymes showed good performance at acid pH's.

TABLE 1

| | | | | Pectin removal |
|---|---|---|---|---|
| pH | Enzyme type | Enzyme Dose | | (%) (average) |
| 4 | No enzyme | 0 | | 24.7 |
| | Acid Pectinase A | 9 | UPTE/g fabric | 46.8 |
| | Acid Pectinase A | 90 | UPTE/g fabric | 61.8 |
| | Acid Pectinase B | 13 | PGU/g fabric | 60.4 |
| | Acid Pectinase B | 130 | PGU/g fabric | 95.6 |
| 3 | No enzyme | 0 | | 24.0 |
| | Acid Alpha- | 130 | PGU/g fabric | 91.2 |
| | Amylase B | | | |

| (ml/kg) | 0 | 0.5 | 5.00 |
|---|---|---|---|
| , pH 4 | 24.7% | 46.8% | 61.8% |
| Pectinex Yield Mash, pH 4 | 24.7% | 47.2% | 79.8% |
| Pectinex Ultra, pH 4 | 24.7% | 60.4% | 95.8% |
| Pectinex XXL, pH 4 | 24.7% | 30.4% | 69.5% |
| Pectinex Smash XXL, pH 4 | 24.7% | 32.9% | 88.9% |
| Pectinex BE XXL, pH 3 | 24.0% | | 91.2% |

### Example 3

### Cold Pad-Batch Simultaneous Desizing and Bioscouring with Acid Amylase A and Acid Pectinase A

The Vilisco fabric (100% cotton) was from Vlisco and cut to 5 cm*15 cm. Buffer pH 3 and pH 4 were prepared followed the procedures described in Example 1. 100 ml buffer was added to a beaker, Keirlon Jet B was added to a concentration of 2 g/L. Enzymes (the doses were listed in Table 2) were added to the impregnation solution and mixed well. Fixed 2 swatches of the same fabric in a pair of forceps. Dip the swatches in the impregnation bath for 30 seconds and pad it with the padder (Mathis Inc, U.S.A.). Repeated dipping and squeezing for one more time to ensure a 100% wet pick-up. Placed the swatches in two layers of plastic bag, pressed out the air and place the bag at room temperature. After 24 hours, removed the samples from the plastic bag. Fixed the samples in the forceps and dipped them in a water bath at 90° C. for 30 seconds and squeeze with padder. Repeated the dipping and squeezing twice. Rinsed the fabric in cold tap water for at least 60 seconds and squeeze off the water by hand. Then airs dry the fabric and measure TEGEWA, residual pectin, wetting time and wicking test. The result of the test was shown in Table 2.

### Example 4

### Pad-Batch Simultaneous Desizing and Bioscouring with Acid Amylase A and Acid Pectinase A

The same fabric and same buffer system were used as Example 3. Added 100 ml impregnation solution to each beaker and placed them in the Lab-o-Mat, heated the solutions to 60° C. Took out the beaker and added enzymes according to Table 2 to the impregnation solution and mixed well. Fixed 2 swatches of the same fabric in a pair of forceps. Dipped the swatches in the impregnation bath for 30 seconds and padded it with the padder. Repeated dipping and squeezing for one more time to ensure a 100% wet pick-up. Placed the swatches in two layers of plastic bag, pressed out the air and placed the bag at the water bath pre-set to 60° C. After 2 hours, removed the samples from the plastic bag. Fixed the samples in the forceps and dipped them in a water bath at 90° C. for 30 seconds and squeezed with padder. Repeated the dipping and squeezing twice. Rinsed the fabric in cold tap water for at least 60 seconds and squeezed off the water by hand. Then air dried the fabric and measured TEGEWA, residual pectin, wetting time and wicking test. The result of the test was shown in Table 2.

TABLE 2

| | | | Desizing | Pectin | Wetting | Wicking |
|---|---|---|---|---|---|---|
| | Amylase A | Pectinase A | (TEGEWA) | removal | time (s) | (cm) |
| Raw fabric | 0 | 0 | 1 | 0 | >60 s | NA |
| Cold Pad-Batch | 50 | 36000 | 7 | 72.6% | 5 | 9 |
| (pH 3); 25° C. | AFAU/L | UPTE/L | | | | |
| Cold Pad-Batch | | | 7 | 68.2% | 6 | 9 |
| (pH 4); 25° C. | | | | | | |
| Pad-Batch | | | 9 | 73.3% | 6 | 9 |
| (pH 3), 60° C. | | | | | | |
| Pad-Batch | | | 6.5 | 69.9% | 4 | 9 |
| (pH 4), 60° C. | | | | | | |

### Example 5

### Cold Pad-Batch Simultaneous Desizing and Bioscouring with Acid Amylase A and Acid Pectinase B

The procedures were the same as described in Example 3 except that Acid Pectinase B was used. The result of the test is shown in Table 3.

### Example 6

### Pad-Batch Simultaneous Desizing and Bioscouring with Acid Amylase A and Acid Pectinase B

The procedures were the same as described in Example 4 except Acid Pectinase B was used. The result of the test is shown in Table 3.

TABLE 3

| | | | Desizing | Pectin | Wetting | Wicking |
|---|---|---|---|---|---|---|
| | Amylase A | Pectinase B | (TEGEWA) | removal | time (s) | (cm) |
| Cold Pad-Batch | 50 AFAU/L | 52000 | 7 | 76.5% | 5 | 10 |
| (pH 3), 25° C. | | PGU/L | | | | |
| Cold Pad-Batch | | | 8 | 75.4% | 2 | 10 |
| (pH 4), 25° C. | | | | | | |
| Pad-Batch | | | 8 | 75.4% | 5 | 11 |
| (pH 3), 60° C. | | | | | | |
| Pad-Batch | | | 6.5 | 72.6% | 4 | 9.5 |
| (pH 4), 60° C. | | | | | | |

### Example 7

### Cold Pad-Batch Simultaneous Desizing and Bioscouring with Acid Amylase B and Acid Pectinase A

The procedures were the same as described in Example 3 except that Acid Amylase

A was replaced by Acid Amylase B. The result of the test is shown in Table 4.

### Example 8

### Pad-Batch Simultaneous Desizing and Bioscouring with Acid Amylase B and Acid Pectinase A

The procedures were the same as described in Example 4 except that Acid Amylase A was replaced by Acid Amylase B. The result of the test was shown in Table 4.

TABLE 4

| | | | Desizing | Pectin | Wetting | Wicking |
|---|---|---|---|---|---|---|
| | Amylase B | Pectinase A | (TEGEWA) | removal | time (s) | (cm) |
| Cold Pad-Batch | 50 FAU/L | 36000 | 9 | 69.9% | 6 | 9.5 |
| (pH 3), 25° C. | | UPTE/L | | | | |
| Cold Pad-Batch | | | 9 | 58.1% | 5 | 8.5 |
| (pH 4), 25° C. | | | | | | |
| Pad-Batch | | | 8.5 | 71.1% | 10 | 10 |
| (pH 3), 60° C. | | | | | | |
| Pad-Batch | | | 9 | 62.1% | 5 | 10 |
| (pH 4), 60° C. | | | | | | |

### Example 9

### Cold Pad-Batch Simultaneous Desizing and Bioscouring with Acid Amylase B and Acid Pectinase B

The procedures were the same as described in Example 3 except that Acid Amylase A was replaced by Acid Amylase B and Acid Pectinase A was replaced by Acid Pectinase B. The result of the test is shown in Table 5.

### Example 10

### Pad-Batch Simultaneous Desizing and Bioscouring with Acid Amylase B and Acid Pectinase B

The procedures were the same as described in Example 4 except that Acid Amylase A was replaced by Acid Amylase B and Acid Pectinase A was replaced by Acid Pectinase B. The result of the test is shown in Table 5.

TABLE 5

| | | | Desizing | Pectin | Wetting | Wicking |
|---|---|---|---|---|---|---|
| | Amylase B | Pectinase B | (TEGEWA) | removal | time (s) | (cm) |
| Cold Pad-Batch | 50 FAU/L | 52000 | 8 | 74.5% | 13 | 9.5 |
| (pH 3), 25° C. | | PGU/L | | | | |
| Cold Pad-Batch | | | 8.5 | 65.7% | 2 | 10 |
| (pH 4), 25° C. | | | | | | |
| Pad-Batch | | | 9 | 75.2% | 4 | 9.5 |
| (pH 3), 60° C. | | | | | | |
| Pad-Batch | | | 7.25 | 69.4% | 9 | 9 |
| (pH 4), 60° C. | | | | | | |

### Example 11

### Desiring Cotton Fabric with Wild-Type Acid Alpha-Amylase A

A 100% cotton fabric (270 g/m²) was from Boräs Wäfveri Kungsfors AS. Sweden. It was made in 2003 with Cupper 3/1 construction The fabric contained 28 thread/cm warp yarn and 14 thread/cm weft yarn. The warp yarn has Ne 11 and the weft has Ne 8. Both yarns were open end. The dry size pick up on the warp yarn was 8%. The size contained mainly Kollotex 5, Solvitose XO, and beef tallow wax with emulsifier. Kollotex 5 is a tow viscous potato starch ester. Solvitose XO is a high viscous starch ether with DS about 0.07. Fabric swatches were cut to about 25 g each.

Buffer pH 3 was made by dissolving 11.53 g 85% phosphoric acid in 4.5 titer pure water, titrating with 5 N NaOH to pH 2.95, then adding water to 5 liter. After adding 2g/l nonionic surfactant (a wetting agent) in the buffer, the buffer pH was measured as 3.05 at 25° C. The dose of enzymes was added as listed in table 6.

The desizing treatment was conducted in a Lab-o-mat (Werner Mathis). A 250 mL buffer solution was added in each beaker, A given amount of alpha-amylase enzyme was added. One fabric swatch (25 g) was placed in each beaker. The beaker was closed and placed in the Lab-o-mat. Beakers were heated at 5° C./min to 50° C. by an infrared heating system equipped within the Lab-o-mat. Beakers were rotated at 30 rpm, 50° C. for 45 minutes. After the enzyme treatment, the fabric swatch was sequentially washed with water in the same beaker three times at 95, 75. and 40° C., respectively.

After dry overnight in air, the fabric swatch was stained with an iodine solution. The stained fabric sample was visually compared to TEGEWA standard photos with 1-9 scale where 1 is dark and 9 has no color stain. Thus higher number indicates a better starch removal. The visual evaluation was done by at least three professionals and an average TEGEWA value was given for each fabric sample. The results are shown in Table 6.

The residue of metal ions on fabric was also evaluated. The fabric was first cut through 1 mm sieve with a Thomas-Wiley mill. Fabric mash 4.00 (+/−A0.01) g was mixed with 80 mL 1 g/L EDTA solution. The mixture was incubated at 70° C. and 200 rpm in a shaker (new Brunswick Scientific Co. Inc Series 25) for 15 hours. After cooled down for about 30 minutes, the mixture was centrifuged at 2500 rpm at 20° C. for 10 minutes. The supernatant was collected for metal content analysis with a Perkinelmer atomic absorption spectrophometer.

TABLE 6

| | [Enzyme] | TEGEWA | | |
|---|---|---|---|---|
| | (AFAU/ | Value | Metal content (mg/L) | |
| Enzyme Type | kg fabric) | (average) | Mn | Fe |
| No enzyme | 0 | 1.3 | 0.23 | 3.91 |
| Acid Alpha- | 27.5 | 2.3 | n/a | n/a |
| Amylase A | 275 | 3.8 | 0.20 | 2.72 |
| | 1100 | 5.2 | n/a | n/a |

n/a = not measured.

1-32. (canceled)

33. A process for combined desizing and scouring of a sized fabric containing starch or starch derivatives during manufacture of a fabric, which process comprises incubating said sized fabric in an aqueous treating solution having a pH in the range between 1 and 7, wherein the aqueous treating solution comprises an acid amylase and at least one acid scouring enzyme.

34. The process of claim 33, wherein the aqueous treating solution has a pH in the range between 1 and 5.

35. The process of claim 33, wherein the scouring enzyme is acid cellulase, acid pectinase, acid lipase, acid xylanase and/or acid protease or a mixture thereof.

36. The process of claim 33, wherein the acid amylase is derived from a strain of Aspergillus, Meripilus, or Rhizomucor.

37. The process of claim 36, wherein the acid amylase is the Aspergillus niger alpha-amylase having an amino acid sequence of SEQ ID NO: 38.

38. The process of claim 36, wherein the acid amylase is the Rhizomucor pusillus alpha-amylase having an amino acid sequence of SEQ ID NO: 48.

39. The process of claim 33, wherein the acid amylase is derived from a strain of Bacillus.

40. The process of claim 33, wherein the acid amylase is derived from a strain of Bacillus amyloliquefaciens, Bacillus licheniformis, Bacillus stearothermophilus, or Bacillus subtilis.

41. The process of claim 33, wherein the acid amylase is derived from Bacillus sp. NCIB 12289, NCIB 12512, NCIB 12513, DSM 9375, DSMZ 12648, DSMZ 12649, KSM AP1378, KSM K36 or KSM K38.

42. The process of claim 33, wherein the scouring enzyme is an acid pectate lyase, an acid pectin lyase, an acid polygalacturonase, and/or an acid polygalacturonate lyase.

43. The process of claim 33, wherein the scouring enzyme is an acid pectinase derived from Aspergillus or Bacillus.

44. The process of claim 33, wherein the process is carried out at a temperature in the range from 5-90° C.

45. The process of claim 44, wherein the process is carried out at a temperature between 25 and 60° C. for a period of between 2 and 24 hours.

46. The process of claim 33, wherein the pH is in the range between pH 2 to 4.

47. The process of claim 33, wherein the fabric is cotton fabric, denim, linen, ramie, viscose, lyocell, or cellulose acetate.

48. The process of claim 33, wherein the fabric is made of fibers of animal origin, in particular silk or wool.

49. The process of claim 33, wherein the fabric is made of polyester fibers of man-made or natural origin, such as poly(ethylene terephthalate) or poly(lactic acid).

50. The process of claim 33, wherein the fabric is made of nylon, acrylic, or polyurethane fibers.

51. The process of claim 33, wherein the fabric is a polyester containing fabric or garment consists of essentially 100% polyester.

52. A composition comprising an acid amylase and an acid scouring enzyme.
